(19) United States
(12) Patent Application Publication
(10) Pub. No.: US 2012/0135094 A1
(43) Pub. Date: May 31, 2012

(54) OREGANO AND MINT ANTI-INFLAMMATORY COMPOSITIONS AND METHODS

(75) Inventors: James E. Simon, Princeton, NJ (US); Diandian Shen, Somerset, NJ (US); Hector Rodolfo Juliani, Highland Park, NJ (US); Qingli Wu, Annandale, NJ (US)

(73) Assignee: RUTGERS, THE STATE UNIVERSITY OF NEW JERSEY, New Brunswick, NJ (US)

(21) Appl. No.: 13/320,292

(22) PCT Filed: May 14, 2010
(86) PCT No.: PCT/US10/34907

§ 371 Date: Feb. 14, 2012

Related U.S. Application Data
(60) Provisional application No. 61/178,199, filed on May 14, 2009.

(51) Int. Cl. A61K 31/19; A61P 29/00; A61K 36/53; A61K 31/216; A61K 36/534

(52) U.S. Cl. 424/745; 514/533; 514/557; 424/747

(57) ABSTRACT

The present invention relates to bioactivity-guided isolation and identification of bioactive compounds from oregano and mint plants, in particular, rosmarinic acid, oleanolic acid and ursolic acid, and use of these compounds or combinations thereof as anti-inflammatory agents for the treatment of conditions related to pain and inflammation and/or as ingredients of dietary supplements. The invention also relates to optimization of the methods for qualitative and quantitative analysis of the bioactive compounds in oregano and mint plants. In particular, this invention introduces an LC/MS (SIM mode) method to achieve co-quantitation of the three organic acids using a unique tandem column system. In addition, the invention also relates to the methods for recovering various water-soluble polyphenols and triterpenes from aromatic plants.

### CROSS REFERENCE TO RELATED APPLICATIONS

This application claims the benefit under 35 U.S.C. §119(e) of U.S. Provisional Application No. 61/178,199, filed on May 14, 2009, which is incorporated herein by reference.

### FIELD OF THE INVENTION

The present invention relates to isolation and identification of bioactive compounds from oregano and mint plants, in particular, rosmarinic acid, oleanolic acid and ursolic acid, and use of these compounds or combinations thereof as anti-inflammatory agents for the treatment of conditions related to pain and inflammation and/or as ingredients of dietary supplements. The invention also relates to optimization of the methods for qualitative and quantitative analysis of the bioactive compounds in oregano and mint plants. In addition, the invention also relates to the methods for recovering various water-soluble polyphenols and triterpenes from aromatic plants at the same time or following the traditional commercial practices by which essential oils are distilled and/or recovered from plants.

### BACKGROUND OF THE INVENTION

Anti-inflammatory drugs are the most frequently prescribed drug class in the world for the treatment of acute or chronic conditions where pain and inflammation are present. (Asero, R., Allergy Asthma Clin. Immunol. 2007, 3, 24-30). These drugs are generally used for the symptomatic relief of conditions such as rheumatoid arthritis, osteoarthritis, inflammatory arthropathies, acute gout, dysmenorrhoea, metastatic bone pain, postoperative pain, pyrexia, renal colic headache and migraine. With their widespread use, the safety concern is of prime importance when they are intended for chronic disease treatment through oral delivery, as evidenced by the withdrawal of several novel prescription non-steroidal anti-inflammatory drugs (NSAIDS) from the market due to potential health risk (Goldkind, L. and Laine, L., Pharmacoepidemiol. Drug Saf 2006, 15, 213-220; Singh, G., J. Rheumatol. 1999, 26 (Suppl), 18-24). However, the adverse effects of these drugs have been recognized. In 2004, rofecoxib was voluntarily removed from the market by the manufacturer due to a potential risk of myocardial infarction and stroke. Valdecoxib was also withdrawn from the market in 2005 because of an increased risk of adverse cardiovascular events in coronary artery surgery trials. Meanwhile, natural dietary supplements with anti-inflammatory activities are gaining interest due to reduced cost and toxicity relative to other treatments.

Oregano (Origanum spp.), an aromatic plant belonging to Origanum genus in the Lamiaceae family and popular as a food seasoning being used since ancient times in southern Europe, is a rich source of aromatic volatile compounds known as essential oils. Greek oregano (O. vulgare ssp. hirtum) represents the most popular oregano species (Adam, K., et al., J. Agric. Food Chem. 1998, 46, 1739-1745). The chemical components in oregano volatile oils have long interested researchers for their aromatic flavoring, antioxidative, antibacterial and antiseptic properties, and this genus is well recognized for its great diversity in aroma and flavor from the myriad of chemotypes available and from which many new varieties have been bred (Kulisic, T., et al., Food Chem. 2004, 85, 633-640; Rodrigues, M. R. A., et al., J. Agric. Food Chem. 2004, 52, 3042-3047; Velluti, A., et al., J. Sci. Food Agric. 2004, 84, 1141-1146). Recently, the water-soluble extract of oregano was reported to inhibit COX-2 secretion showing anti-inflammatory activity in human epithelial carcinoma cells (Lemay, M., Anti-Inflammatory Phytochemicals: In Vitro and Ex Vivo Evaluation. In Phytochemicals, Meskin, M. S.; Bidlack, W. R.; Randolph, R. K., eds.; CRC Press LLC: Boca Raton, Fla., 2006, 41-60). Yoshino et al. found that oregano extract exhibited anti-inflammatory activities in mouse models of stress-induced gastritis and contact hypersensitivity (Yoshino, K., et al., J. Health Sci. 2006, 52, 169-1731). Moreover, the effect of methanol and aqueous methanol extract of O. vulgare ssp. hirtum on soybean lipoxygenase was noted, revealing a promising potential of oregano for anti-inflammatory efficacy (Koukoulitsa, C., et al., Phytother. Res. 2006, 20, 605-606). However, these anti-inflammatory studies were all performed on oregano crude extracts without any information as to what compounds may be responsible, and no research to date has brought these into a molecular level, linking the bioactivities to specific compounds from oregano extracts to the reported anti-inflammatory activity. Moreover, the biomass following essential oil distillation of oregano and other aromatic plants, such as mints (peppermints and spearmints), has long been viewed as waste products used as soil amendments, composted, or in some regions even dried and later used as fuel to fire and charge the next distillation or fed to animals. In addition the water associated with the spent plant material that had been distilled and the water underneath the plant material left in the distillation chamber (also known as a retort, tub, container) that was not part of the steam vapor that carries off the natural essential oils (aromatic volatiles oils) into a condenser which later reverts from gaseous liquid phase into water and oil, has also been viewed and treated as a waste product and has been discarded, drained onto the soil, discarded in some manner, and even rinsed and washed away during the cleaning of the distillation unit.

Since ancient times, mints have been the popular aromatic plants used for food flavoring, teas, liqueur and medicines (Regina, K., et al., J. Ethnopharmacol. 2007, 109, 248-257). As perennial herbs, mints may be strikingly variable with respect to morphology and chemical composition, notably in their essential oils, and relative to other natural products. Mints are one of the most cultivated plants for essential oils, including peppermint (Mentha x piperita and other Mentha spp.) and spearmint (Mentha spicata and M. cardiaca) as the two major crops followed by Japanese Mint (M. arvensis) (Simon, J. E., Essential Oils and Culinary Herbs, in Advances in New Crops; Janick, J., Simon, J. E., eds.; Timber Press: Portland, Oreg., 1990, 472-483). The volatile oils of peppermint and of spearmint are the main reasons for its popular use, and the uses of natural mint oils or synthetic mint flavoring for the flavoring oral care, confectionery and chewing gum are of growing importance (Guntert, M., et al., Flavor Chemistry of Peppermint Oil (Mentha Piperita L.), in Aroma Active Compounds in Foods-Chemistry and Sensory Properties, Takeoka, G. R.; Guntert, M.; Engel, K. H., eds.; American Chemical Society: Washington, DC, 2001, 119-137). The estimated production of peppermint oil in the United States was about 5,000 metric tons (Guntert, M., et al., 2001). The essential oil is typically found in concentrations from 0.3 to 0.4% in peppermint, while for some varieties it may reach as high as 1.5%. Menthol is the main active constituent for peppermint, followed by menthone, menthyl acetate, and over 100 other different compounds in the oil (Guntert, M., et al., 2001). The essential oil profiles of other mints can be different from peppermint For example, the typical spearmint oil usually contains carvone as the major ingredient in the oil (55-67%) and also contains limonene (2-25%), while the other constituents (menthone, menthol, menthofuran, menthyl acetate, and cineole) are expected to be less than 2%. Japanese mint is the natural source of commercial menthol, as that compound is the primary constituent found in its essential oil (Simon, J. E., Essential Oils and Culinary Herbs, in Advances in New Crops; Janick, J., Simon, J. E., eds.; Timber Press: Portland, Oreg., 1990, 472-483).

Traditionally, mints are used to treat common cold, functional dyspepsia, and skin itching (Bruneton, J., Pharmaconosy Phytochemistry Medicinal Plants, Lavoisier Publishing Inc.: Secaucus, N.Y., 1995, 431-437). Recent research reported their antioxidant, anticancer and anti-inflammatory properties, and being clinically effective in alleviating the nasal symptoms of allergic rhinitis (Thiagarajan, D., et al., FASEB J. 2001, 15, A630; Triantaphyllou, K., et al., Int. J. Food Sci. Nutr. 2001, 52, 313-317; Inoue, T., et al., Bio. Pharm. Bull. 2001, 24, 92-95; Takahashi, A. and Nakata, K., Aromatopia 1995, 13, 42-45). The most frequently reported Mentha spp. and consequently the most widely used as traditional medicines include four taxa: M. x piperita and M. spicata, M. arvensis, followed by others such as M. pulegium, and M. longifolia. The musty smelling mints (M. spicata and M. suaveolens) were described restrictedly used for common cold and cough, while the medicinal uses proposed for the pungent smelling herbs may include the treatment of stomach, digestion and respiratory ailments (Regina et al., 2007). Mint oil distilled from leaves is extensively used in the cosmetics and food industries for its antifungal, antimicrobial, insecticidal and antioxidant properties (Bruneton, J., Pharmaconosy Phytochemistry Medicinal Plants, Lavoisier Publishing Inc.: Secaucus, N.Y., 1995, 431-437).

In addition to the commonly recognized bioactivities, some other biological effects may also be present in mint. The anti-inflammatory activity of mints is not found in all Mentha spp., but appears to be limited to several taxa including M. piperita, M. suaveolens and M. aquatica. M. piperita was reported to show anti-inflammatory effects against acute (xylene-induced ear oedema in mice) and chronic (cotton-pellet granuloma .in rats) inflammation (Atta, A. H. and Alkofahi, A., J. Ethnopharmacol. 1998, 60, 117-124). Moreno et al. found the methanol extract from M. suaveolens possessed an anti-inflammatory action inhibiting the carrageenin-induced rat 1Saw oedema (Moreno, L., et al., Phytother. Res. 2002, 16, 310-313). In a research of pharmacological screening of Mediterranean diets, the alcoholic extract of mint (M. aquatica) was described to exhibit inhibition on cytokine-induced cell activation on nitrite assays (Anon, Pharmacol. Res. 2005, 52, 353-366). More recently, Conforti, F., et al. have described that the alcoholic extract Of M. aquatica showed topical anti-inflammatory activity on oedema reduction (Conforti, F., et al., J. Ethnopharmacol. 2008, 116, 144-151). The relation between the anti-inflammatory activity and those compounds that may be responsible had not been explored in depth, nor have there been reports on the genetic effluence both between and within Mentha genus, an aromatic herb that is well known for a diverse range of chemotypes, particularly with the essential oils.

Flavonoids, widely distributed in the plant kingdom, have been recognized as important nutrients for human health (Merken, H. M. and Beecher, G. R., Analysis of Flavonoids in Botanical: A Review, in Quality Management of Nutraceuticals; Ho, C. T.; Zheng, Q. Y., eds.; American Chemical Society: Washington, D.C., 2002, 21-41). These compounds are considered antioxidants, scavenging free radicals by donating hydrogen, and agents preventing oxidation of low-density lipoproteins oxidation. Numerous health benefits have been reported for flavonoids, including heart disease prevention, anti-AIDS, anti-arthritic, anticancer, anti-hypertensive, anti-inflammatory, and antiviral activities (Arpentine, G., et al., Process Group Polyphenols 1992, 16, 237-240; Frankel, E. N., et al., Lancet 1993, 341, 1103-1104; Hu, J. P., et al., Biological Trace Element Res. 1995, 47, 327-331; Khokhar, S., et al., Cancer Lett. 1997, 114, 171-172; Terencio, M. C., et al., J. Ethnopharmacol. 1991, 31, 109-114; Meunier, M. T., et al., Planta Med. 1987, 53, 12-15; Kuo, S. M., Ontogenesis 1997, 8, 47-69). Flavonoid glycosides from peppermint have been previously identified as luteolin-7-O-glucoside, luteolin-7-O-rutinoside, apigenin-7-O-glucoside, isorhoifolin, hesperidin, eriocitrin, piperitoside, menthoside diosmetin, and diosmin (Guedon, D. J. and Pasquier, B. P., J. Agric. Food Chem. 1994, 42, 679-684; Areias, F. M., et al., Food Chem. 2001, 73, 307-311; Hoffmann, B. G. and Lunder, L. T., Planta Med., 1984, 50, 361; Yamanura et al., 1998; Subramanian, S. S, and Nair, A. G. R., Phytochemistry 1972, 11, 452-453). Free flavonoid aglycones, including luteolin, apigenin, and acacetin, were found in peppermint (Justesen, U., J. Chromatogr. A 2000, 902, 369-379; Voirin, B., et al., Biochem. Syst. Ecol. 1994, 22, 95-99; Voirin, B. and Bayet, C., Phytochemsitry 1992, 31, 2299-2304; Zakharov, A. M., et al., Khimiya Prirodnykh Soedinenii 1987, 1, 143-144; Voirin, B., et al., Phytochemistry 1999, 50, 1189-1193) and exhibited anti-allergic, anti-inflammatory and choleretic activities (Malialal, P. P. and Wanwimolruk, S., J. Pharm. Pharmacol. 2001, 53, 1323-1329; Takahashi, A. and Nakata, K., Aromatopia 1995, 13, 42-45; Nair, B., Int. J. Toxicol. 2001, 20, 61-73). The phenolic profiles in members of this genus, outside of the popular main essential oil varieties or sources of peppermint and spearmint, have not been systematically explored and surveyed.

In particular, rosmarinic acid is an ester of caffeic acid and 3,4-dihydroxyphenyllacetic acid, a phenolic compound found in many culinary herbs within the Lamiaceae family, and has been reported in Mentha species as well as other plants in Lamiaceae family including basil (Ocimum spp.), rosemary (Rosmarinus spp.) and thyme (Thymus spp.) (Peterson, M. and Simmonds, M. S. J., Phytochemistry 2003, 62, 121-125). Several papers reported its presence in Origanum spp. and its antioxidative, anti-inflammatory and anti-depressive activities (Exarchou, V., et al., J. Agric. Food Chem. 2002, 50, 5294-5299; Hideyuki, M., et al., Biosci. Biotechnol. Biochem. 2003, 67, 2311-2316; Takeda, H., et al., Eur. J. Pharmacol. 2002, 449, 261-267). This phenolic compound was described as a free radical scavenger and inhibitor of low-density lipoprotein oxidation (Nakamura, Y., et al, J. Agric. Food Chem. 1998, 46, 4545-4550; Fuhrman, B., et al., Antioxid. Redox Signal. 2000, 2, 491-506), while no one related this compound to the anti-inflammatory activities of mint. The anti-inflammatory properties were described by the inhibition of lipoxygenases and cyclooxygenases (Pamham, M. J. and Kesselring, K., Drugs Future 1985, 10, 756-757; Yamamoto, H., et al., J. Agric. Food Chem. 1998, 46, 862-865).

Oleanolic acid and its isomer ursolic acid are triterpenoids which exist in the plant kingdom as free acids or in the conjugated form known as triterpenoid saponins (Liu, J., J. Ethnopharmacol. 1995, 49, 57-68). The compound oleanolic acid has been patented in Japan as a health-promoting additive to drinks, and marketed in China as a safe non-prescription drug for treatment of liver disorders (Chen, L., et al., Bioorg. Med. Chem. Lett. 2007, 17, 2979-2982). Many triterpenoids possess anti-inflammatory effects, and oleanolic acid and ursolic acid are among the most notable bioactive triterpenoids (Price, K. R., et al.; Crit. Rev. Food Sci. Nutr. 1987, 26, 27-135; Mahato, S. B., et al., Phytochemistry 1988, 27, 3037-3067). The anti-inflammatory mechanism of oleanolic acid and ursolic acid is postulated as simultaneously affecting multiple targets in one or more signaling pathways (Dai, Y., et al., Acta Pharmacol. Sin. 1989, 10, 381-384; Tsuruga, T., et al., Chem. Pharmacol. Bull. 1991, 39, 3276-3278; Simon, A., et al., Biochim. Biophys. Acta 1992, 1125, 68-72; Najid, A., et al., FEBS J. 1992, 299, 213-217; Zhou, C., et al., J. Clin. Pharmacol. Sci. 1993, 2, 69-79; Ying, Q. L., et al., Biochem. J. 1991, 277, 521-526; Dai, Y., et al., Chin. J. Pharmacol. Toxicol. 1989, 3, 96-99; Kapil, A. and Shanna, S., J. Pharm. Pharmacol. 1994, 46, 922-923). Oleanolic acid and ursolic acid were reported to be present in several Mentha species including M. arvensis, M. spicata, M. rotundifolia and M. vulgare (Karasawa, D. and Shimizu, S., Agric. Biol. Chem. 1980, 44, 1203-1205; Perva, A., et al., Isolation of Active Components of Lamiaceae, 1. Zbornik Referatov s Posvetovanja Slovenski Kemijski Dnevi, Maribor, Slovenia 2001, 856c/1-856c/7; Hadolin, M., et al., Isolation of active components of Lamiaceae, I. Zbornik Referatov s Posvetovanja Slovenski Kemijski Dnevi, Maribor, Slovenia 2001, 856b/1-856b/7); however, due to lack of analytical survey of these triterpenoid acids in Mentha spp., how much these contents contribute to the hydroalcoholic extract of mints are still unclear.

Several papers described the qualitative or quantitative determination of water-soluble counterpart in mint species (Kosar, M., et al., J. Agric. Food Chem. 2004, 52, 5004-5010). Guedon et al. developed an HPLC method to analyze plant samples by using a Nucleosil C18 column with water (pH=2.5) and acetonitrile as a gradient eluent, resulting in flavonoid glycosides and rosmarinic acid quantitation (Guedon, D. J. and Pasquier, B. P., J. Agric. Food Chem. 1994, 42, 679-684). The screening of free radical scavenging compounds in water extracts of Mentha samples was performed by using a postcolumn DPPH assay (Kosar, M., et al., J. Agric. Food Chem. 2004, 52, 5004-5010). In the present inventors' work, seven flavonoids plus caffeic acid and rosmarinic acid were quantitated in mint samples. However, many of the flavonoids determined in these samples were flavonoid aglycones. The more complex flavonoid glycosides such as luteolin-7-O-rutinoside, narirutin, hesperidin and diosmin, naturally present as the predominant flavonoid forms, still remain to be further examined and approved through different Mentha spp. More recently, four phenolic compounds luteolin-7-β-glucuronide, caffeic acid, rosmarinic acid, and lithospermic acid were quantitatively determined in medicinal herbs including mint by using HPLC/UV with a C18 column and acetonitrile-water-formic acid mobile phase (Fecka, I., et al., Chromatographia 2007, 66, 87-93). All references cited in this application are incorporated by reference in their entirety.

Recent surveys show that approximately 50% of the US population use dietary supplements at one time or another, and that annual sales in the dietary supplement industry were over $18 billion. The Food and Drug Administration (FDA) defines a dietary supplement as “a product (other than tobacco) that is intended to supplement the diet and bears or contains one or more of the following dietary ingredients: a vitamin, a mineral, a herb or other botanical, an amino acid, or a dietary substance for use by man to supplement the diet by increasing the total daily intake, or a concentrate, metabolite, constituent, extract, or combinations of these ingredients.” Additionally, there is a significant market for supplement and feed additives for the livestock, poultry and pet industries. The global feed additive market was estimated as 6 billion dollars in 2000, covering the products containing vitamins, amino acids, growth enhancers, carotenoids, antioxidants and enzymes. The worldwide animal feed additives market is expected to reach US $15.4 billion by 2010, of which the disease-preventing agents are valued to be $485 million. With the antibiotics widely used in feed additives, the strict regulatory environment is one of the biggest challenges facing the animal feed additives markets, and the development of natural plant-based feed additive containing phytopharmaceuticals are gaining interest.

In particular, equine arthritis is a major concern of horse owners and is becoming more common as horses are living longer. Current treatments include allowing the animal to rest, pursuing physical therapy such as ice or heat treatments, and receiving anti-inflammatory agents injected into the muscles or affected joints. The development of anti-inflammatory feed additives from natural plants is expected to be an emerging market for animal health. Thus, there is a need for development of new natural anti-inflammatory agents for the treatment of adverse conditions in humans and animals.

### SUMMARY OF THE INVENTION

In response to the foregoing need, the present invention provides extracts of the culinary herbs and spices, Oregano and Mint, as sources of anti-inflammatory compositions and dietary supplements. The invention also provides methods for the preparation of these extracts and isolation of the organic acid compounds from these plants, which are responsible for the biological activities of the plants.

In one aspect the present inventors identified the anti-inflammatory agents in oregano as rosmarinic acid, oleanolic acid and ursolic acid by using bioactivity-guided isolation. A quantitative survey of these anti-inflammatory constituents in different oregano species (O. vulgare ssp. hirtum, O. vulgare and O. syriacum) and chemotypes within the species demonstrates that the correct chemotype of this plant is a significantly rich source of rosmarinic, oleanolic and ursolic acids. Significant variation in chemical composition between species and within a species was found.

In one aspect the present invention provides an anti-inflammatory composition derived from an oregano or mint plant, or any species of the Lamiaceae family, the composition comprising at least one organic acid selected from the group consisting of rosmarinic acid, oleanolic acid and ursolic acid.

In another aspect the present invention provides a dietary supplement comprising an anti-inflammatory composition derived from an oregano or mint plant, or any species of the Lamiaceae family, the composition comprising at least one organic acid selected from the group consisting of rosmarinic acid, oleanolic acid and ursolic acid.

In another aspect the present invention provides a method for treating an inflammatory condition in a human or animal subject, the method comprising administering to the subject a therapeutically effective amount of an anti-inflammatory composition derived from an oregano or mint plant, or any species of the Lamiaceae family, the composition comprising at least one organic acid selected from the group consisting of rosmarinic acid, oleanolic acid and ursolic acid.

The inventors observed a synergistic action for the combination of these three compounds (rosmarinic acid:oleanolic acid:ursolic acid=2:1:2) on the LPS-induced nitrite production assay. The oregano samples from Zatar, also called Syrian Oregano (a unique species) from Origanum syriacum, contain distinctly high contents of these three anti-inflammatory compounds, and as a result exhibited the highest anti-inflammatory activities among other Origanum species and varieties when adjusted to a per gram (g) dry weight basis.

In another aspect the present invention provides a method for separation and isolation of organic acids, in particular, rosmarinic acid, oleanolic acid, and ursolic acid from an oregano or mint sample.

In another aspect the present invention provides a method for co-quantitation of rosmarinic acid, oleanolic acid and ursolic acid in an oregano or mint sample, the method comprising use of a tandem high performance liquid chromatography (HPLC) system comprising a first stationary phase and a second stationary phase, wherein the first and the second stationary phases are different from each other.

In another aspect the present invention provides a method for quantitatively determining the content of rosmarinic acid, oleanolic acid and/or ursolic acid in an oregano or mint sample, the method comprising use of an LC/MS/MS system in a multiple reaction monitoring (MRM) mode, wherein rosmarinic acid is monitored by selecting m/z 359 as the parent ion and m/z 161 as the daughter ion, and wherein oleanolic acid and ursolic acid are each monitored by selecting m/z 439 as the parent ion and m/z 203 as the daughter ion.

The present inventors developed and validated an HPLC method for co-quantitation of the anti-inflammatory constituents in mints. Based on the LC/MS method on oreganos, the inventors further developed a LC/MS/MS method for the simultaneous quantitation of the three anti-inflammatory acids in mints The methods have been successfully used to analyze the contents of flavonoid compounds in different mints, which contribute to a major part of phytochemicals in mints and possess numerous health-promoting properties. Thirty-five different mint varieties and accessions (coming from 9 Mentha species collected from around the world) were examined for their chemical profiles. An HPLC/UV method on the determination of the major flavonoid contents in mints was developed and validated herein.

In another aspect the present invention provides a method for recovering water-soluble polyphenol and/or triterpene compounds from an oregano or mint plant, comprising extracting the plant material with a hot water or water vapor to obtain an aqueous phase enriched by polyphenol and/or triterpene compounds and isolating the polyphenol and/or triterpene compounds from the enriched aqueous phase, wherein the extracting step optionally occurs in tandem with a distillation step.

This recovery process facilitates a framework for the concentration of bioactive compounds in these plants when the actual concentration is far lower than desirable, as is often the case with natural products. The compositions (extracts or purified compounds) derived from the during- or post-distillation materials can be commercialized as dietary supplements or as anti-inflammatory agents to reduce pain and discomfort from inflammation and other health indications that arise from inflammatory conditions. These products can be placed into and used in human foods and supplements as well as animal feed additives including, but not limited to, those for the treatment of equine rheumatic arthritis. Therefore, the method not only provides an efficient means to exploit the aromatic plants to the full extent in discovering and obtaining biologically important compounds but also provides tremendous commercial value for manufacture of these bioactive compounds from the plants while minimizing the waste.

### BRIEF DESCRIPTION OF THE DRAWINGS

FIG. 1 illustrates the chemical structures of the anti-inflammatory compounds, namely rosmarinic acid, oleanolic acid, and ursolic acid.

FIG. 2 illustrates the scheme of the dietary supplement product development using oregano as the case study, yet recognizing a similar scheme using mint and mint extracts could be substituted for oregano. Furthermore, we state that similar schemes using other species than the genus Origanum and Mentha and/or other members of the Lamiaceae family such as basil (Ocimum spp.), catnip (Nepeta cataria), catmint (Nepeta spp.), lavender and lavendin (Lavandula spp.), perilla (Perilla frustescens), rosemary (Rosmarinus officinalis), sage (Salvia spp.), and thyme (Thymus spp.) can also be achieved in the same or like manner.

FIG. 3 illustrates the mass spectra of (A) rosmarinic acid (negative), (B) oleanolic acid (positive), and (C) ursolic acid (positive), obtained from the LC/MS TIC of oregano extract on an electrospray ionization (ESI) mass spectrometer.

FIG. 4 illustrates anti-inflammatory effect of rosmarinic acid (RosA), ursolic acid (UA) and oleanolic acid (OA) on LPS-induced nitrite production in RAW 264.7 macrophages. The cells were treated with 100 ng/mL of LPS only or with different concentrations of test compounds for 24 h. At the end of incubation time, 100 μL of the culture medium was collected for nitrite assay. The values are expressed as means±standard error of triplicate tests.

FIG. 5 illustrates effects of rosmarinic acid (RosA), oleanolic acid (OA), ursolic acid (UA) and indomethacin on LPS-induced iNOS and COX-2 protein levels in RAW 264.7 cells. The cells were treated with test compounds (10 and 20 μg/mL) for 24 h. Equal amounts of total proteins (50 μg) were subjected to 10% SDS-PAGE. The expression of iNOS, COX-2 and β-actin protein was detected by Western Blot using specific antibodies. Quantification of iNOS and COX-2 protein expression was performed by densitometric analysis of the immunoblot. The values below the figure represent the relative intensity in protein expression of the bands normalized to β-actin.

FIG. 6 illustrates an SIM (selected ion monitoring) chromatogram of (A) standards and representative chromatograms of (B) sample EO1 (Origanum vulgare), (C) sample SO6 (O. syriacum), and (D) sample GO2 (O. vulgare ssp. hirtum). The two time segments were set as 0-10 min at m/z 359 (rosmarinic acid) and 10-40 min at m/z 479 (oleanolic acid and ursolic acid).

FIG. 7 illustrates the mass spectra of (A) rosmarinic acid (negative), (B) oleanolic acid (positive), and (C) ursolic acid (positive) obtained from the LC/MS TIC of mint extract (Mentha x piperita) on an electrospray ionization (ESI) mass spectrometer.

FIG. 8 illustrates a proposed MS fragmentation pathway of rosmarinic acid.

FIG. 9 illustrates a proposed MS fragmentation pathway of ursolic acid.

FIG. 10 illustrates MRM (multiple reaction monitoring) chromatogram of (A) standards and representative chromatograms of (B) Peppermint (Mentha x piperita), (C) Lavender Mint (M. aquatica), (D) Persian Mint (M. x piperita), and (E) Orange Mint (M. aquatica). The two time segments were set as 0-10 min at m/z 359-161 (rosmarinic acid), and 10-40 min at m/z 439-203 (oleanolic acid and ursolic acid).

FIG. 11 illustrates a TIC (Total Ion Chromatogram) of a mint sample (Peppermint, Mentha x piperita). For peak identities, see Table 6.

FIG. 12 illustrates the chemical structures of the major phenolic compounds in mint (Mentha spp.).

FIG. 13 illustrates an HPLC/UV chromatogram of the phenolic standards (A), and 3 representative chromatograms of mint samples, namely Fuzzy Spearmint (Mentha spicata) (B), Peppermint (M. x piperita) (C), and Lime Mint (M. aquatica x M. suaveolens) (D), monitored at wavelength of 280 nm.

FIG. 14 illustrates a flow diagram for the recovery of polyphenol and/or triterpene compounds from spent oregano or mint plant material or from an aqueous waste following distillation.

### DETAILED DESCRIPTION OF THE INVENTION

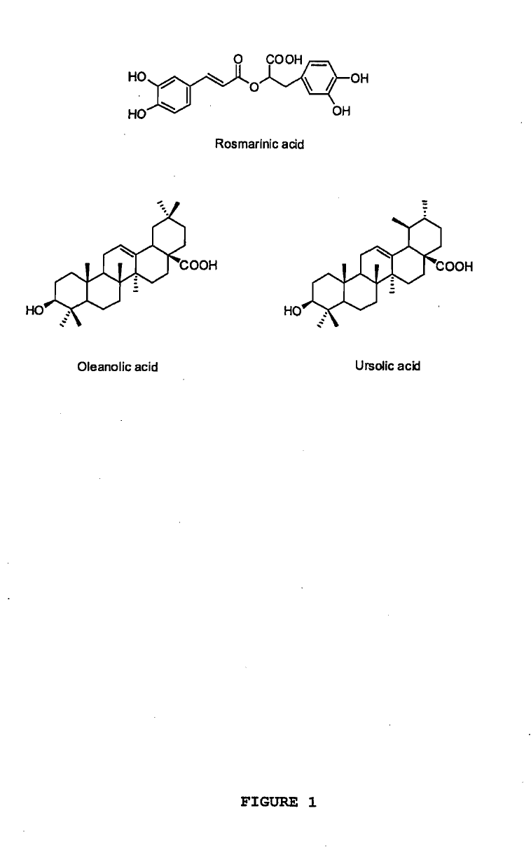

Using a bio-directed fractionation approach the present inventors successfully identified the anti-inflammatory constituents in oregano as rosmarinic acid, oleanolic acid and ursolic acid (FIG. 1). The present inventors are the first to demonstrate that these compounds are responsible for the anti-inflammatory property of oregano. The inventors conducted a quantitative survey of these anti-inflammatory chemical constituents in different types, varieties and species of Oregano (Origanum species). The results showed that oregano could be a significantly rich natural source of rosmarinic acid, oleanolic acid and ursolic acid. Rosmarinic acid was the predominant compound in the varieties of Origanum vulgare ssp. hirtum and Origanum vulgare, ranging from 13.73 mg/g to 63.69 mg/g on dry weight basis in leaves. The average levels of oleanolic acid and ursolic acid in these two species were 1.96 mg/g and 6.72 mg/g, calculated from twenty-two different varieties. The varieties of Origanum syriacum (Zatar and Syrian Oregano) showed a distinctly high content of triterpenoid acids, with oleanolic acid averaging 9.40 mg/g in seven different varieties and ursolic acid averaging 24.07 mg/g.

These three compounds identified in oregano were tested on the LPS-induced nitrite production assay and the Western Blotting of LPS-induced iNOS and COX-2 protein levels in murine cells, all showing stronger or comparable anti-inflammatory activities compared to the control indomethacin, a recognized anti-inflammatory agent (Ismaili, H., et al., J. Pharm. Pharmacol. 2001, 53, 1645-1652). Subsequently, the present inventors found the combination of the three or two ingredients at certain percentage may bring the activity to an even higher level of potency on nitrite production assay. The inventors' observations indicate that: (i) oregano has anti-inflammatory activity; (ii) this activity is linked to the three compounds the inventors have identified; (iii) not all three of these compounds contributed equally to this anti-inflammatory activity; and (iv) it is possible to develop dietary supplements from the post-distillation material of oregano or fresh or dried materials that can be enriched and thus serve as a source of anti-inflammatory agents. In addition, these discoveries lead us to recognize that each of these compounds and/or their combinations, regardless of plant origin or plant source, could serve as anti-inflammatory agents. From these observations, the inventors can conclude that oregano, particularly those chemotypes with a high concentration of these bioactive compounds, could be suitable candidates for development of a dietary supplement product as a potential complimentary and alternative phytomedicine.

Thus, in one aspect the present invention provides an anti-inflammatory composition derived from an oregano or mint plant, or any species of the Lamiaceae family, the composition comprising at least one organic acid selected from the group consisting of rosmarinic acid, oleanolic acid and ursolic acid.

In one embodiment of this aspect, the present invention provides an anti-inflammatory composition derived from a species selected from Origanum vulgare ssp. hirtum, Origanum vulgare, and Origanum syriacum.

In one preferred embodiment of this aspect, the present invention provides an anti-inflammatory composition derived from a species of Origanum syriacum.

In another embodiment of this aspect, the present invention provides an anti-inflammatory composition derived from a Mentha spp. selected from the group consisting of M. x piperita, M. spicata, M. aquatica, M. x villosa, M. x gracilis, M longifolia, M. aquatica x M. suaveolens, M. canadensis, and M. x smithiana.

In one preferred embodiment of this aspect, the present invention provides an anti-inflammatory composition derived from a Mentha spp. selected from the group consisting of peppermint (M. x piperita), spearmint (M. spicata and M. cardiaca), lavender mint (M. aquatica), Persian mint (M. x piperita), chewing gum mint (M. x piperita), orange mint (M. aquatica), apple mint (M.x villosa), Austrian mint (M.x gracilis), balsam tea mint (M. x piperita), chocolate mint (M. x piperita), curly mint (M. spicata), Egyptian mint (M. x villosa), fuzzy spearmint (M. spicata), grapefruit mint (M. x piperita), green curly mint (M. x piperita), Hajek mint (M. longifolia), Hillary's sweet mint (M. aquatica x M. suaveolens), Hypocalyx mint (M. Canadensis), Japanese field mint (M. Canadensis), lime mint (M. aquatica x M. suaveolens), regular mint (M. spicata), Scotch mint (M. x gracilis), Todd Mitcham mint (M. x piperita), variegated mint (M. x piperita), and water mint (M. x smithiana).

In another preferred embodiment of this aspect, the present invention provides an anti-inflammatory composition derived from a Mentha spp. selected from green curly mint (M. x piperita), spearmint (M. spicata), fuzzy spearmint (M. spicata), grapefruit mint (M. x piperita), Japanese field mint (M. Canadensis), Egyptian mint (M. x villosa), Hillary's sweet mint (M. aquatica x M. suaveolens), and apple mint (M.x villosa).

In another preferred embodiment of this aspect, the present invention provides an anti-inflammatory composition comprising rosmarinic acid, oleanolic acid and ursolic acid in a 2:1:2 ratio by weight.

In another aspect the present invention provides a dietary supplement comprising an anti-inflammatory composition derived from an oregano or mint plant, or any species of the Lamiaceae family, the composition comprising at least one organic acid selected from the group consisting of rosmarinic acid, oleanolic acid and ursolic acid.

In one preferred embodiment of this aspect, the present invention provides a dietary supplement comprising an anti-inflammatory composition comprising rosmarinic acid, oleanolic acid and ursolic acid in a 2:1:2 ratio by weight.

In another aspect the present invention provides a method for treating an inflammatory condition in a human or animal subject, the method comprising administering to the subject a therapeutically effective amount of an anti-inflammatory composition derived from an oregano or mint plant, or any species of the Lamiaceae family, the composition comprising at least one organic acid selected from the group consisting of rosmarinic acid, oleanolic acid and ursolic acid.

In one preferred embodiment of this aspect, the present invention provides a method for treating an inflammatory condition in a human or animal subject, the method comprising administering to the subject a therapeutically effective amount of an anti-inflammatory composition comprising rosmarinic acid, oleanolic acid and ursolic acid in a 2:1:2 ratio by weight.

The inventors observed a synergistic action for the combination of these three compounds (rosmarinic acid:oleanolic acid:ursolic acid=2:1:2) on the LPS-induced nitrite production assay. The oregano samples from Zatar, also called Syrian Oregano (a unique species) from Origanum syriacum, contain distinctly high contents of these three anti-inflammatory compounds, and as a result exhibited the highest anti-inflammatory activities among other Origanum species and varieties when adjusted to a per gram (g) dry weight basis.

In another preferred embodiment of this aspect, the animal subject is a horse, and the inflammatory condition is equine rheumatic arthritis.

In another aspect the present invention provides a method for separation and isolation of an organic acid, in particular, rosmarinic acid, oleanolic acid, and ursolic acid from an oregano or mint sample, the method comprising the steps of:

(a) extracting the oregano or mint sample with an organic solvent to obtain an organic extract and a solid residue;

(b) optionally separating the solid residue from the organic extract;

(c) evaporating the organic solvent from the organic extract to obtain a liquid residue enriched with the organic acid;

(d) isolating the organic acid from the liquid residue by silica gel or ion-exchange chromatography eluting with a gradient of organic solvents until the subject organic acid is eluted out in eluent fraction(s);

(e) evaporating the eluting solvents from the fraction(s) containing the subject organic acid obtained in step (d); and

(f) optionally repeating steps (d) and/or (e) until the subject organic acid is completely separated from other components of the liquid residue.

In one preferred embodiment of this aspect, the present invention provides a method for isolation of an organic acid, in particular, rosmarinic acid, oleanolic acid, and ursolic acid from an oregano or mint sample, wherein the oregano or mint sample is in the form of a dry powder.

In one preferred embodiment of this aspect, the present invention provides a method for isolation of organic acids, in particular, rosmarinic acid, oleanolic acid, and ursolic acid, from an oregano or mint sample, wherein the organic solvent(s) in the extracting step comprises a C₁-C₆ alcohol.

In one preferred embodiment of this aspect, the present invention provides a method for isolation of an organic acid, in particular, rosmarinic acid, oleanolic acid, and ursolic acid from an oregano or mint sample, wherein the organic solvent(s) in the extracting step comprises methanol or ethanol.

In one preferred embodiment of this aspect, the present invention provides a method for isolation of an organic acid, in particular, rosmarinic acid, oleanolic acid, and ursolic acid from an oregano or mint sample, wherein the method further comprises validation steps of (g) spiking the oregano or mint sample with a known quantity of the subject organic acid standard, and (h) measuring the total amount of the subject organic acid in the mixture.

In one preferred embodiment of this aspect, the present invention provides a method for isolation of an organic acid from an oregano or mint sample, wherein the organic acid is rosmarinic acid.

In one preferred embodiment of this aspect, the present invention provides a method for isolation of an organic acid from an oregano or mint sample, wherein the organic acid is oleanolic acid.

In one preferred embodiment of this aspect, the present invention provides a method for isolation of an organic acid from an oregano or mint sample, wherein the organic acid is ursolic acid.

In another aspect the present invention provides a method for co-quantitation of rosmarinic acid, oleanolic acid and ursolic acid in an oregano or mint sample, the method comprising use of a tandem high performance liquid chromatography (HPLC) system comprising a first stationary phase and a second stationary phase, wherein the first and the second stationary phases are different from each other.

In one preferred embodiment of this aspect, the present invention provides a method for co-quantitation of rosmarinic acid, oleanolic acid and ursolic acid in an oregano or mint sample, wherein the first stationary phase comprises an ether-linked phenyl phase with a polar end-capping so as to increase the retention time of rosmarinic acid through π-π interactions between the aromatic rings of rosmarinic acid and the phenyl group of the stationary phase, and the second stationary phase comprises an octadecyl silane (ODS) phase.

In one preferred embodiment of this aspect, the present invention provides a method for co-quantitation of rosmarinic acid, oleanolic acid and ursolic acid in an oregano or mint sample, wherein the first stationary phase comprises a Phenomenex® Synergi Polar-RP column, and the second stationary phase comprises a Varian® Microsorb ODS column.

In one preferred embodiment of this aspect, the present invention provides a method for co-quantitation of rosmarinic acid, oleanolic acid and ursolic acid in an oregano or mint sample, wherein the first stationary phase consists of a Phenomenex® Synergi Polar-RP 50×4.6 mm column, and the second stationary phase consists of a Varian® Microsorb ODS 100×4.6 mm column.

In another aspect the present invention provides a method for quantitatively determining the content of rosmarinic acid, oleanolic acid and/or ursolic acid in an oregano or mint sample, the method comprising use of an LC/MS/MS system in a multiple reaction monitoring (MRM) mode, wherein rosmarinic acid is monitored by selecting m/z 359 as the parent ion and m/z 161 as the daughter ion, and wherein oleanolic acid and ursolic acid are each monitored by selecting m/z 439 as the parent ion and m/z 203 as the daughter ion.

The present inventors developed and validated an HPLC method for co-quantitation of the anti-inflammatory constituents in mints. Based on the LC/MS method on oreganos, the inventors further developed a LC/MS/MS method for the simultaneous quantitation of the three anti-inflammatory acids in mints. The methods have been successfully used to analyze the contents of flavonoid compounds in different mints, which contribute to a major part of phytochemicals in mints and possess numerous health-promoting properties. Thirty-five different mint varieties and accessions (coming from 9 Mentha species collected from around the world) were examined for their chemical profiles. An HPLC/UV method on the determination of the major flavonoid contents in mints was developed and validated herein.

In another aspect the present invention provides a method for recovering water-soluble polyphenol and/or triterpene compounds from an oregano or mint plant, comprising extracting the plant material with a hot water or water vapor to obtain an aqueous phase enriched by polyphenol and/or triterpene compounds and isolating the polyphenol and/or triterpene compounds from the enriched aqueous phase, wherein the extracting step optionally occurs in tandem with a distillation step.

In one embodiment of this aspect, the present invention provides a method for recovering water-soluble polyphenol and/or triterpene compounds from an oregano or mint plant, the recovery method further comprising a step of separating the enriched aqueous phase from an oil phase that may be formed.

In one embodiment of this aspect, the present invention provides a method for recovering water-soluble polyphenol and/or triterpene compounds from an oregano or mint plant, wherein the extracting step further comprises extracting the plant material with a water-immiscible organic solvent to obtain an organic phase enriched with oil compounds and separating the polyphenol and/or triterpene enriched aqueous phase from the organic phase.

In one embodiment of this aspect, the present invention provides a method for recovering water-soluble polyphenol and/or triterpene compounds from an oregano or mint plant, wherein the extracting step occurs in tandem with a distillation step, and wherein the distillation step removes volatile essential oils from the plant material.

In one embodiment of this aspect, the present invention provides a method for recovering water-soluble polyphenol and/or triterpene compounds from an oregano or mint plant, wherein the extracting step occurs in tandem with a distillation step, and wherein after distillation the spent plant material is further extracted with a revert current water vapor, hot water or solvent to obtain an additional amount of extract enriched with the polyphenol and/or triterpene compounds.

In one embodiment of this aspect, the present invention provides a method for recovering water-soluble polyphenol and/or triterpene compounds from an oregano or mint plant, the method further comprising a step of quantifying the remaining content of the polyphenol and/or triterpene compounds in the plant material.

In one embodiment of this aspect, the present invention provides a method for recovering water-soluble polyphenol and/or triterpene compounds from an oregano or mint plant, the method further comprising a step of quantifying the total amount of phenols remaining in the plant material by using the Folin-Ciocalteu method.

In one embodiment of this aspect, the present invention provides a method for recovering water-soluble polyphenol and/or triterpene compounds from an oregano or mint plant, wherein the enriched aqueous solution is an aqueous waste from a distillation process.

In one embodiment of this aspect, the present invention provides a method for recovering water-soluble polyphenol and/or triterpene compounds from an oregano or mint plant, wherein the isolating step comprises removal of water by evaporation, freeze drying, spin chromatography, differential boiling, or spray drying, or any other equivalent technique.

In one embodiment of this aspect, the present invention provides a method for recovering water-soluble polyphenol and/or triterpene compounds from an oregano or mint plant, further comprising separation of the polyphenol and/or triterpene compounds by chromatography.

Moreover, the present inventors developed chromatographic methods, including silica gel and ion-exchange chromatography, to enrich the anti-inflammatory components from less concentrated oregano and mint plant materials. This part of the discovery process facilitates a framework for the concentration of such bioactive compounds in these plants when the actual concentration is far lower than desirable, as is often the case with natural products. The compositions (extracts or purified compounds) developed from the post-distillation materials can be commercialized as dietary supplements or as anti-inflammatory agents to reduce pain and discomfort from inflammation and other health indications that arise from inflammatory conditions. These products can be placed into and used in human foods and supplements as well as animal feed additives including, but not limited to, those for the treatment of equine rheumatic arthritis. Therefore, the method not only provides an efficient means to exploit the aromatic plants to the full extent in discovering and obtaining many biologically important compounds but also provides tremendous commercial value for the manufacture of these compounds through minimizing the waste.

An anti-inflammatory composition of the present invention or a dietary supplement comprising the composition is suitable for treatment of acute or chronic diseases or disorders in humans or other mammalian animals associated with inflammation. A dietary supplement of the present invention may be ingested, for example, administered orally or intragastrically. In some cases, it can be administered by other routes, such as nasally, intravenously, intramuscularly, subcutaneously, sublingually, intrathecally, or intradermally. Any amount of a dietary supplement provided herein can be administered to a mammal, and the dosages of a dietary supplement may depend on many factors, including the mode of administration.

A dietary supplement of the present invention can be in the form of a tablet, capsule, pill, gelcap, powder, liquid, solution, suspension, or gel. For oral administration, tablets or capsules can be prepared by conventional means in combination with physiologically acceptable excipients, such as fillers, binding agents, lubricants, disintegrants, and/or wetting agents, and the tablets can be coated, if desired. Liquid preparations for oral administration can take the form of, for example, solutions, syrups, or suspension, or they can be presented as a dry product for constitution with saline or other suitable liquid vehicle before use. Dietary supplements of the type described herein may also contain acceptable additives such as suspending agents, emulsifying agents, non-aqueous vehicles, preservatives, buffer salts, flavoring, coloring, and sweetening agents as appropriate. Preparations for oral administration may also be formulated to give controlled release of the ingredients.

In some cases, a dietary supplement of the present invention can contain an acceptable carrier for administration to a mammal (e.g., a human or a horse), including, without limitation, sterile aqueous or non-aqueous solutions, suspensions, and emulsions. Aqueous carriers include, but are not limited to, water, saline, buffered solutions, or combinations thereof. Examples of non-aqueous solvents include, without limitation, lower alkyl alcohols, propylene glycol, polyethylene glycol, vegetable oils, and injectable organic esters. Acceptable carriers may also include physiologically acceptable aqueous vehicles (e.g., saline) or other known carriers appropriate to specific routes of administration.

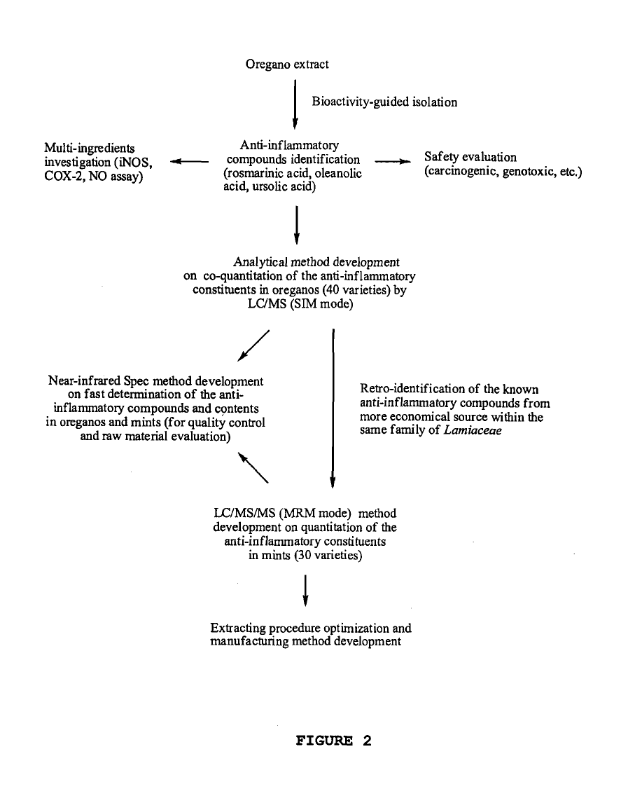

In addition, the invention also encompasses a process for preparation of dietary supplements comprising any one of the bioactive compounds identified from oregano and/or mint plants, in particular, rosmarinic acid, oleanolic acid, ursolic acid, or combinations thereof, or an extract enriched with these compounds derived from oregano and/or mint plants. The process is generally outlined in the scheme illustrated in FIG. 2.

The discovery of the compounds in oregano that are largely responsible for the plant's antioxidant activity and anti-inflammatory action, as well as the research that shows this species to be a rich source of rosmarinic acid, oleanolic acid and ursolic acid, have inspired the present inventors to look for additional sources of these natural anti-inflammatory agents within the same family of Lamiaceae, including a variety of Mentha species.

Thus, the illustrative, non-limiting examples described in more detail below fall into, inter alia, the following three interrelated aspects: (a) analysis of oregano plants, (b) analysis of mint plants, both of which include method development for identification and separation of bioactive compounds, in particular, rosmarinic acid, oleanolic acid and ursolic acid contained in these plant materials, and (c) recovery of the bioactive polyphenol and triterpene compounds from the plant materials, including spent plant material, which is the plant material that has been subjected to distillation or another technique to recover the essential oils, and aqueous waste after distillation to remove volatile essential oils.

### A. Analysis of Oregano

Few papers have described the simultaneous quantitation of oleanolic acid and ursolic acid (Chert, J. H., et al., J. Pharm. Biomed. Anal. 2003, 32, 1175-1179; Altinier, G., et al., J. Agric. Food Chem. 2007, 55, 1718-1723), since these two organic acids are positional isomers, and the only difference between the two is a single methyl group on ring E (Me-30 versus Me-29), which makes their separation for analytical purposes challenging. As a person of ordinary skill in the art would know, the selectivity of MS detection is based on the molecular ion and/or the fragments of analytes, while collision induced dissociation of MS detection provides identical molecular ion and fragmentation patterns for these two isomers. Therefore, the selectivity of MS separation under selected ion monitoring (SIM) or multiple reaction monitoring (MRM) mode can not be achieved unless they are base line separated on the HPLC column Rosmarinic acid differs largely from these two triterpenoid acids relative to chemical polarity, and to date no HPLC method has been reported for the quantification of these three organic acids together in a single run. Therefore, the present work aimed to develop an analytical method to simultaneously quantitate these anti-inflammatory organic acids which was finally achieved by using a tandem column system coupled with MS detector.

The inventors collected and grew in a single location a wide germplasm collection of different oregano species and varieties, purchased commercially available oregano as well that originated in other countries, and quantitatively analyzed the anti-inflammatory constituents by the analytical method developed. Results show that Origanum spp. may contain significantly high concentration levels of rosmarinic acid, oleanolic acid and ursolic acid, and that significant differences in the accumulation of each compound was noted among the accessions and commercial products evaluated.

### Identification of Rosmarinic Acid, Oleanolic Acid and Ursolic Acid in Oregano by LC/MS and NMR Techniques

The inventors successfully developed an LC/MS (MRM mode) method to achieve co-quantitation of these three organic acids with the application of a unique tandem column system. The detection of rosmarinic acid was optimal under negative ion mode of SIM, while oleanolic acid and ursolic acid were sensitive to positive ion mode. The simultaneous quantitation was attained by setting two time segments in one run to facilitate the ESI polarity switch. For the investigated analytes rosmarinic acid, oleanolic acid and ursolic acid, good linearities (r²>0.999) were obtained for each calibration curve. Validation for this method showed a precision (relative standard deviation) ranging from 4.84% to 6.41%, and the recoveries varied from 92.2% to 100.8% for the three analytes.

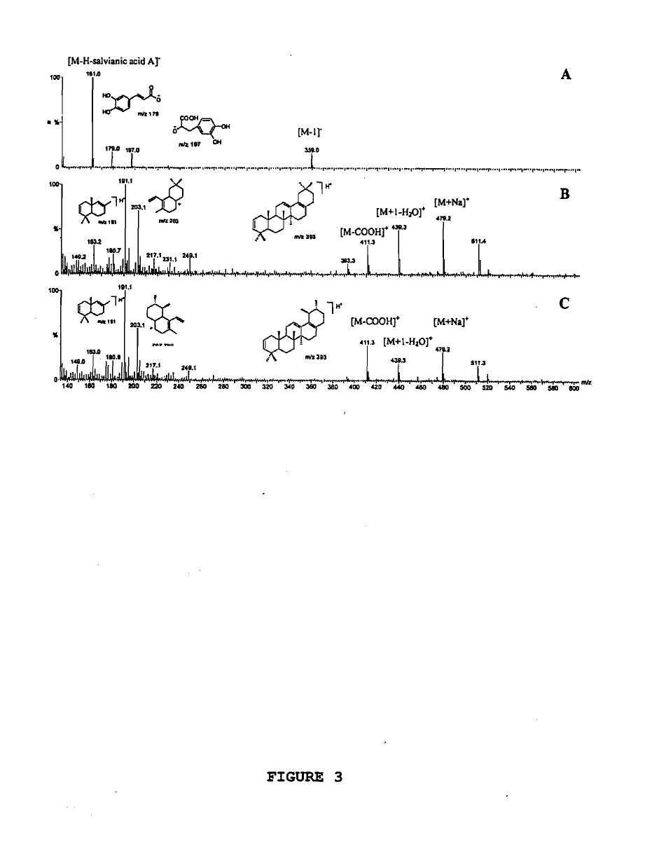

In this invention, full-scan LC/MS spectra under negative ion mode provided molecular ion at m/z 359 ([M−H]⁻), and fragmentation ions at m/z 197 ([salvianic acid A —H]⁻), m/z 179 ([M−H—caffeic acid]) and m/z 161 ([M−H—salvianic acid A]) for rosmarinic acid (FIG. 3). The comparison of the retention time with the commercial reference compound on HPLC further supported the identification of rosmarinic acid in our oregano samples. Oleanolic acid and ursolic acid have been isolated from more than 120 plant species (Liu, J., J. Ethnopharmacol. 1995, 49, 57-68), but never before have been reported in Origanum spp. Oleanolic acid and ursolic acid both produce identical molecular ions and fragmentations on mass spectra. The ion peaks at m/z 479 were the sodium adduct molecular ions [M+Na]+ of oleanolic acid and ursolic acid. Dehydration (m/z 439) and decarboxylation acid (m/z 411) products were observed as MS fragments. The fragmentation ions at m/z 191 and m/z 203 were due to RDA reactions, the characteristic MS fragmentation of 4¹²-unsaturated triterpenoids. The structures of oleanolic acid and ursolic acid were further elucidated by NMR data at the stage of isolated pure compounds (Table 1), and compared with literature (Seto, H., et al., Agric. Biol. Chem. 1986, 50, 939-942; Ahmad, V. U., et al., J. Nat. Prod. 1993, 56, 329-334; Huang, P., et al.; Wang, L., J. Nat. Prod. 1999, 62, 891-892; Alves, J. S., et al., Magn. Reson. Chem. 2000, 38, 201-206).

TABLE 1
¹³C and ¹H NMR Assignments for Oleanolic Acid and
Ursolic Acid (in Pyridine-d₅)

| | Oleanolic acid | | | Ursolic acid | | |
|---|---|---|---|---|---|---|
| Position | δ¹³C | δ¹H | | δ¹³C | δ¹H | |
| 1 | 37.8 | | | 38.3 | | |
| 2 | 27.0 | | | 27.1 | | |
| 3 | 77.0 | 3.44 | m | 77.1 | 3.46 | m |
| 4 | 38.3 | | | 38.0 | | |
| 5 | 54.7 | 083 | brs | 54.8 | | |
| 6 | 17.7 | | | 17.7 | | |
| 7 | 32.1 | | | 32.5 | | |
| 8 | 38.7 | | | 38.9 | | |
| 9 | 47.0 | | | 47.0 | | |
| 10 | 36.3 | | | 36.2 | | |
| 11 | 22.6 | | | 22.6 | | |
| 12 | 122.9 | 5.49 | brs | 124.6 | 5.49 | brs |
| 13 | 143.6 | | | 138.2 | | |
| 14 | 41.1 | | | 41.5 | | |
| 15 | 27.6 | | | 27.6 | | |
| 16 | 22.7 | | | 23.9 | | |
| 17 | 45.5 | | | 47.0 | | |
| 18 | 40.9 | | | 52.5 | 2.64 | d |
| | | | | | (J = 11.2 Hz) | |
| 19 | 45.4 | | | 38.3 | | |
| 20 | 29.8 | | | 38.4 | | |
| 21 | 33.1 | | | 30.0 | | |
| 22 | 32.1 | | | 36.4 | | |
| 23 | 27.2 | 1.27 | s | 27.8 | 1.24 | s |
| 24 | 15.4 | 1.02 | s | 15.5 | 1.02 | s |
| 25 | 14.4 | 0.88 | s | 14.6 | 0.89 | s |
| 26 | 16.3 | 1.02 | s | 16.4 | 1.05 | s |
| 27 | 25.0 | 1.23 | s | 22.9 | 1.22 | s |
| 28 | 179.3 | | | 178.9 | | |
| 29 | 32.2 | 0.93 | s | 16.5 | 1.00 | d |
| | | | | | (J = 7.2 Hz) | |
| 30 | 22.6 | 1.00 | s | 20.4 | 0.94 | d |

(J = 5.6 Hz)

### Anti-inflammatory Bioassays on Rosmarinic Acid, Oleanolic Acid and Ursolic Acid in Oregano

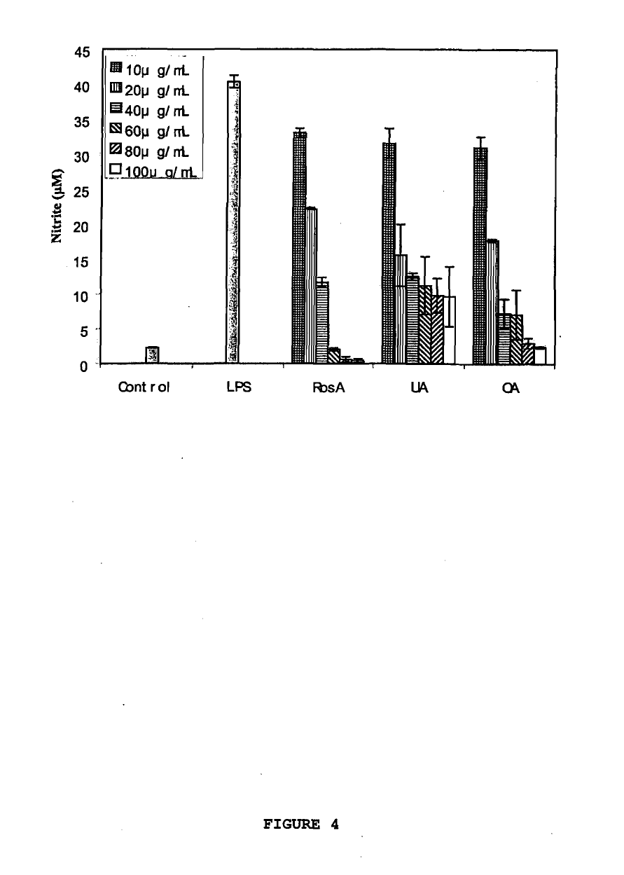

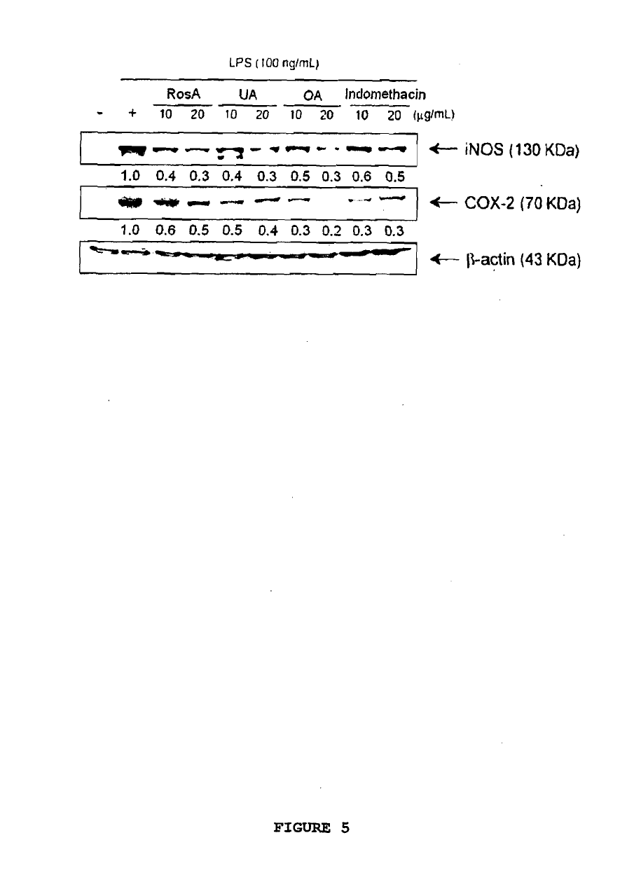

Stimulation of RAW 264.7 cells with LPS results in NO (nitric oxide) accumulation in the media. The nitrite assay measures the amount of nitrite generation, an indicator of NO synthesis. Rosmarinic acid, at a concentration range of 10-100 μg/mL, dose-dependently, suppressed NO generation by 19-100% (FIG. 4). Similar results were obtained for ursolic acid and oleanolic acid, indicating their inhibition of NO production by >50% at the 20 μg/mL concentration level. COX-2 and iNOS protein expression of rosmarinic acid, ursolic acid and oleanolic acid was then observed from Western Blotting (FIG. 5). The reduced expression of iNOS protein by these compounds was consistent with reductions on the nitrite production assay. In addition, these three compounds also suppressed COX-2 protein expression on Western Blotting, showing comparable anti-inflammatory activities contrasting to indomethacin, a recognized COX inhibitor.

### Analytical Method Optimization for Oregano Samples

Separation of organic acids can be difficult due to the retention time shift, peak broadening, and inability to achieve adequate resolution on the chromatogram. The challenge of co-quantitation for this research resides on the chemical similarity of oleanolic acid and ursolic acid, and the drastic difference of polarity between rosmarinic acid and the two triterpenoid acids. The inventors found that an older type ODS column (Microsorb C18) provided improved peak resolution between these two position isomers probably due to the silanol interactions present on the stationary phase Ammonium formate as the buffer in mobile phase at slightly basic condition (pH 7.4) was optimal to achieve sharp peaks, a baseline separation of oleanolic acid and ursolic acid, and ideal retention-time stability. Additionally, the use of ammonium formate enhanced the signal response and improved the sensitivity for MS detection. Finally, the co-quantitation of rosmarinic acid with oleanolic acid and ursolic acid was achieved by employing a tandem column system using two different stationary phases: first a Synergi Polar-RP column from Phenomenex (50×4.6 mm) and downstream a Microsorb ODS column from Varian (100×4.6 mm) Polar-RP column is an ether-linked phenyl phase with polar endcapping, used to increase retention times of highly polar compounds and offer selective retention on aromatic compounds by π-π interactions between the aromatic rings of the analyte and the phenyl functional group of Synergy polar-RP. Methanol, a protic solvent with pronounced hydrogen bonding ability, was found to be superior for chromatographic resolution compared to acetonitrile when combining with water as the mobile phase. Also, aromatic selectivity is further enhanced by the presence of methanol in the mobile phase on Synergy polar-RP column due to the ability of methanol to facilitate π-π interactions between the aromatic rings of rosmarinic acid and the phenol functional group of the stationary phase. In contrast, the c electrons of the “CN” bond in acetonitrile are presumed to compete for the phenyl binding sites on the stationary phase (Yang, M., et al., J. Chromatogr. A 2005, 1097, 124-129). Although rosmarinic acid, oleanolic acid and ursolic acid are all organic acids, they perform differently under electrospray ionization. Rosmarinic acid is more sensitive for ionization under negative mode, whereas the optimal ionization condition for oleanolic acid and ursolic acid is under positive ion mode. The polarity switch as attained by creating two time segments in a single run where the polarity mode changed from negative (0-10 min) to positive (10-40 min).

### Validation of the LC/MS Method for Oregano Samples

The precision of this method was validated by carrying out six replicate determinations of a single oregano sample on the same day (intra-assay) and six analytical batches on three different days (inter-assay). The relative standard deviations (RSDs) of intra-assay were 6.16%, 5.37% and 4.84% for rosmarinic acid, oleanolic acid and ursolic acid, respectively. Inter-assay RSDs were 6.22%, 6.41% and 5.31% for those same analytes. The recoveries of this method were evaluated by the addition of known concentrations of the standards, rosmarinic acid, oleanolic acid and ursolic acid at three concentration levels, approximately 100%, 75% and 50% of the expected values in the oregano sample G012 (O. vulgare ssp. hirtum). No considerable difference was found among the recoveries at different concentration levels, with RSDs being 3.0%, 3.4% and 2.0% for 100%, 75% and 50% spiked levels respectively (Table 2). The mean recoveries were calculated as 98.2% for rosmarinic acid, 97.4% for oleanolic, and 94.2% for ursolic acid. These developed analytical method was reliable, validation studies showed that our newly precise and sensitive for the simultaneous quantitation of rosmarinic acid, oleanolic acid and ursolic acid in oregano samples.

TABLE 2
Recoveries of Rosmarinic Acid, Oleanolic Acid and Ursolic Acid
at Different Spiking Levels

| | Concentra- | | | | | |
|---|---|---|---|---|---|---|
| | tion | Added | Found | Recovery | Mean | RSD |
| Analyte | (g/kg) | (g/kg) | (g/kg) | (%) | (%) | (%) |
| Rosmarinic | 48.63 | 44.40 | 93.36 | 100.7 | | |
| acid | | 33.30 | 80.24 | 94.9 | 98.2 | 3.0 |
| | | 22.20 | 70.57 | 98.8 | | |
| Oleanolic | 2.50 | 2.07 | 4.59 | 100.8 | | |
| acid | | 1.55 | 4.01 | 97.2 | 97.4 | 3.4 |
| | | 1.04 | 3.47 | 94.1 | | |
| Ursolic | 8.48 | 9.32 | 17.30 | 94.6 | | |
| acid | | 6.99 | 14.92 | 92.2 | 94.3 | 2.0 |
| | | 4.66 | 12.95 | 95.9 | | |

### Quantitative Survey of Rosmarinic Acid, Oleanolic Acid and Ursolic Acid Contents in Different Oregano Varieties

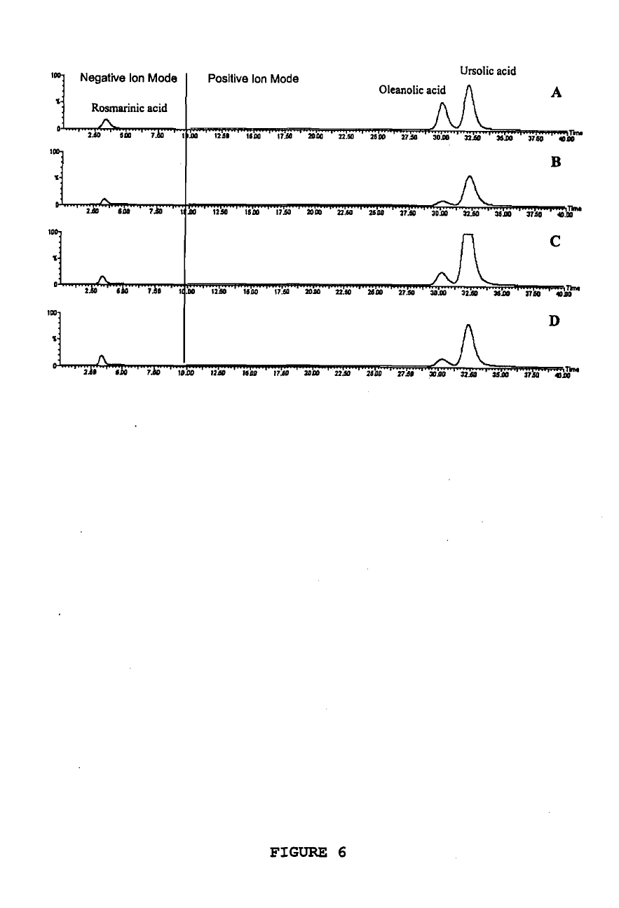

Different sources, varieties and even species of oregano were compared for the accumulation of these three anti-inflammatory constituents. Several oregano sources were commercially purchased as dry aerial parts directly for analysis; others were vegetatively transplanted to our field research station, with parent plants being maintained and grown in greenhouses. This part of the analysis was not to definitively compare growing conditions or ‘sources’ per se but to first ask whether chemical differences in these three bioactive acids were found in oregano, and if so, then comparison could be more strongly examined within the sub-groups of oregano from each source (e.g., within the commercial products; or within the cultivated species and breeding lines). Most of the varieties belonged to O. vulgare ssp. hirtum, O. vulgare and O. syriacum, which are among the major species of Origanum that enter into the global trade (Dogan, S., et al., Food Chem. 2005, 91, 341-345). Based on the LC/MS (SIM mode) method developed (FIG. 6), nearly thirty different oregano sources were quantitatively analyzed for rosmarinic acid, oleanolic acid and ursolic acid content (Table 3).

TABLE 3
Contents of the Anti-inflammatory Compounds in Different Oregano
Varieties (Milligrams per Gram of Dry Matter)

| Sample code^{a} | Rosmarinic acid^{b} | Oleanolic acid^{b} | Ursolic acid^{b} |
|---|---|---|---|
| O. vulgare ssp. hirtum | | | |
| GO1 | 42.56 ± 1.38 | 1.56 ± 0.07 | 5.01 ± 0.11 |
| GO2 | 58.32 ± 0.47 | 3.70 ± 0.23 | 8.59 ± 0.36 |
| GO3 | 43.62 ± 3.05 | 1.48 ± 0.07 | 6.65 ± 0.33 |
| GO4 | 40.32 ± 0.38 | 1.47 ± 0.07 | 4.83 ± 0.11 |
| GO5 | 40.37 ± 3.39 | 2.18 ± 0.03 | 6.86 ± 0.31 |
| GO6 | 37.02 ± 1.24 | 1.90 ± 0.14 | 7.24 ± 0.51 |
| GO7 | 26.12 ± 1.87 | 2.36 ± 0.08 | 8.32 ± 0.48 |
| GO8 | 39.66 ± 0.76 | 2.05 ± 0.15 | 8.34 ± 0.43 |
| GO9 | 63.69 ± 4.54 | 1.07 ± 0.10 | 4.96 ± 0.42 |
| GO10 | 35.01 ± 1.81 | 1.49 ± 0.05 | 5.91 ± 0.46 |
| GO11 | 45.88 ± 0.19 | 1.65 ± 0.11 | 5.80 ± 0.19 |
| GO12 | 48.63 ± 1.81 | 2.50 ± 0.12 | 8.48 ± 0.26 |
| GO13 | 50.94 ± 1.96 | 1.55 ± 0.11 | 5.36 ± 0.21 |
| GO14 | 41.16 ± 1.69 | 1.43 ± 0.10 | 4.70 ± 0.17 |
| GO15 | 62.33 ± 5.19 | 2.26 ± 0.13 | 8.11 ± 0.30 |
| GO16 | 53.24 ± 0.32 | 1.57 ± 0.12 | 5.33 ± 0.19 |
| O. vulgare | | | |
| EO1 | 19.97 ± 0.99 | 2.89 ± 0.13 | 7.78 ± 0.56 |
| EO2 | 14.65 ± 0.79 | 1.99 ± 0.14 | 6.35 ± 0.12 |
| EO3 | 14.49 ± 1,29 | 1.82 ± 0.10 | 7.72 ± 0.23 |
| EO4 | 32.41 ± 2.10 | 1.75 ± 0.11 | 6.66 ± 0.21 |
| EO5 | 13.73 ± 1.17 | 2.96 ± 0.18 | 10.60 ± 0.35 |
| EO6 | 14.17 ± 0.93 | 1.47 ± 0.09 | 4.28 ± 0.13 |
| O. syriacum | | | |
| SO1 | 9.13 ± 0.18 | 9.84 ± 0.11 | 23.84 ± 0.74 |
| SO2 | 35.73 ± 3.42 | 9.87 ± 0.57 | 24.65 ± 0.87 |
| SO3 | 27.65 ± 1.21 | 11.03 ± 0.52 | 34.42 ± 0.91 |
| SO4 | 30.60 ± 1.13 | 12.72 ± 0.05 | 29.47 ± 0.71 |
| SO5 | 24.57 ± 0.30 | 8.30 ± 0.08 | 22.04 ± 0.38 |
| SO6 | 40.37 ± 2.03 | 6.99 ± 0.38 | 17.94 ± 0.21 |
| SO7 | 16.02 ± 0.27 | 7.08 ± 0.32 | 16.12 ± 0.21 |

^{a}GO 1-16 are the varieties of Greek oregano (O. vulgare ssp. hirtum), EO 1-6 are the varieties of European oregano (O. vulgare) and SO 1-7 are the varieties of Syrian oregano (O. syriacum).
^{b}Mean value 4—SD in duplicate.

Many Origanum varieties were found to be an extraordinarily rich source of the anti-inflammatory constituents, although the variation was large within the same species. Rosmarinic acid was the predominant compound in the varieties of O. vulgare ssp. hirtum and O. vulgare, ranging from 13.73 mg/g to 63.69 mg/g on dry weight basis. The average levels of oleanolic acid and ursolic acid in these two species were 1.96 mg/g and 6.72 mg/g, as calculated from twenty-two different species and varieties. The sources of O. syriacum showed a distinct feature of a high content level of triterpenoid acids, with oleanolic acid averaging 9.40 mg/g in seven different sources and ursolic acid averaging 24.07 mg/g. Cuban oregano (Plectranthus amboinicus), a Caribbean native and while having the same common name belonging to another genus in Lamiaceae family, was also collected and analyzed by the same LC/MS method as a comparison as an anticipated negative control purposefully included for analytical purposes. Results showed that none of the three analytes were detected in the aerial part of Cuban oregano, demonstrating again it is distinct from the true members of Origanum. Results of our data allow us to conclude that rosmarinic acid, oleanolic acid and ursolic acid are major nonvolatile second metabolites found in Origanum spp. These compounds accumulated in the aerial part and the distribution varied significantly by species and source. The inventors also conclude that oregano can serve as a rich source of these bioactive anti-inflammatory compounds.

### B. Analysis of Mints

The discovery of oregano as a rich source of rosmarinic acid, oleanolic acid and ursolic acid inspired the inventors to evaluate other members of this family of essential oil bearing plants, i.e., the Lamiaceae, to determine if they would also exhibit anti-inflammatory activity due to the same or similar non-volatile polyphenols. The present inventors examined a wide array of Mentha (mint) species and discovered the presence of these same three anti-inflammatory organic acids (rosmarinic acid, oleanolic acid and ursolic acid) identified by LC/MS.

The present inventors grew 35 different mints from among 9 species, including many major commercial varieties used in the large-scale production and distillation of peppermint and spearmint for the national and international essential oil markets. Growth occurred under identical environmental conditions (both in the field and greenhouse) to allow for observation of differential genetic expression among the mints During the harvest season, the mints were collected and dried under low temperature (32° C.), and samples of 25 of these mints were then quantitatively analyzed for rosmarinic acid, oleanolic acid and ursolic acid based on the LC/MS/MS method which the inventors developed. The average rosmarinic acid content was 10.34 mg/g on dry weight basis across the 25 mints, with RSD=58.6%. The most rosmarinic acid-rich sample was Green Curly Mint (M. x piperita), followed by Spearmint (M. spicata).

### Optimization of Chromatographic Conditions and ESI-MS Parameters for Determination of Rosmarinic Acid, Oleanolic Acid and Ursolic Acid in Mints

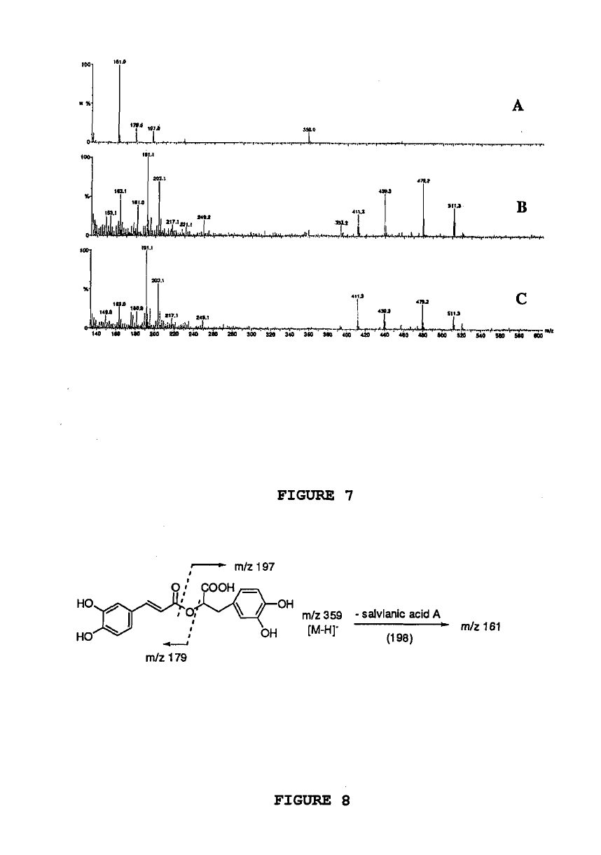

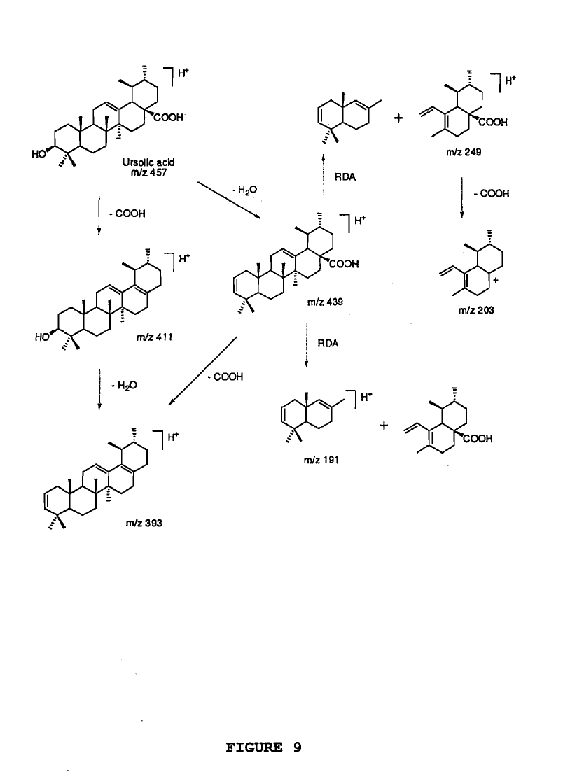

The co-quantitation of rosmarinic acid, oleanolic acid and ursolic acid is difficult due to the chemical similarity of oleanolic acid and ursolic acid, and the drastic difference of polarity between rosmarinic acid and the two triterpenoid acids. The retention time shift and peak broadening also cause failure to quantitate on the chromatogram, especially for the separation of theses organic acids. To achieve simultaneous quantitation of rosmarinic acid with oleanolic acid and ursolic acid, the inventors utilized a tandem column system composed of two different stationary phases: first a Synergi Polar-RP column from Phenomenex (50×4.6 mm) and downstream a Microsorb ODS column from Varian (100×4.6 ram). Polar-RP column, an ether-linked phenyl phase with polar endcapping, applied as the upstream column to selectively increase the retention time of rosmarinic acid by π-π interactions between the aromatic rings of the analyte and the phenyl functional group of Synergy Polar-RP Ammonium formate was finally chosen as the buffer in mobile phase because it provided sharper peaks and baseline separation of oleanolic acid and ursolic acid, ideal retention-time stability, and enhanced sensitivity under MS detection. ESI mass spectra in both negative and positive modes were examined for the detection of analytes. Under negative ion mode, rosmarinic acid presented a better signal-to-noise ratio on mass spectra. The peak at m/z 359 was designated as the molecular ion [M−H]⁻ (FIG. 7). The fragmentation ions at m/z 197 ([salvianic acid A-H]⁻) and m/z 179 ([M−H—caffeic acid]⁻) were also observed (FIG. 8). The base peak at m/z 161 on the spectrum was the fragmentation ion [M−H—salvianic acid A]⁻, which was subsequently selected as the daughter ion being monitored in the third quadrupole. Along with the deprotonated molecule at m/z 359 chosen as the parent ion, the MRM mode provided sensitive detection for rosmarinic acid and good linearity (r²≈0.999) covering the expected concentration range in all mint samples. Oleanolic acid and ursolic acid showed almost identical mass spectra under positive ion mode, with peaks at m/z 479 designated as the sodium adduct molecular ions [H+Na]⁺ and same fragmentation patterns. The [M+H]⁺ at m/z 457 was hardly observed, while the dehydration ion at m/z 439 was abundant and being an important intermediate for the subsequent RDA fragmentations such as product ions at m/z 191 and m/z 203, the characteristic MS fragmentations of Δ¹²-unsaturated triterpenoids (FIG. 9). During the optimization of tandem mass conditions, when ion peak at m/z 439 was utilized as the precursor ion with the downstream fragmentation ion at m/z 203 being the daughter ion, it showed to be sensitive and specific for the detection of oleanolic acid and ursolic acid in the mint samples. The simultaneous quantitation of rosmarinic acid under negative ion mode and triterpenoid acids under positive ion mode was accomplished by creating two time segments in a single run where the polarity mode was changed from negative (0-10 min) to positive (10-40 min)

### LC/MS/MS Method Validation for Mint Samples

The precision of this method was validated by intra-assay and inter-assay evaluations. The relative standard deviations (RSDs) of intra-assay were calculated as 4.68%, 2.36% and 4.89% for rosmarinic acid, oleanolic acid and ursolic acid, respectively, conducted by carrying out six replicate injections of a single mint sample within the same day (Table 4). The inter-assay validation was applied with six replicate determinations of the same mint sample on three different days, and the RSDs were 5.51%, 4.21% and 5.34% for these three analytes. The recoveries of this method were performed by addition of known concentration levels, approximate 200%, 100% and 50% of the expected values in a spearmint sample (M. spicata). The mean recovery rates were 98.7%, 99.3% and 97.8% for rosmarinic acid, oleanolic acid and ursolic acid, respectively, without considerable difference at different spiking levels (RSD<4.6%). This validation study suggested that the developed LC/MS/MS method was reliable, precise and sensitive for the simultaneous quantitation of rosmarinic acid, oleanolic acid and ursolic acid in mint samples.

TABLE 4
Recoveries of Rosmarinic Acid, Oleanolic Acid and Ursolic Acid
at Different Spiking Levels

| | Concentra- | | | | | |
|---|---|---|---|---|---|---|
| | tion | Added | Found | Recovery | Mean | RSD |
| Analyte | (g/kg) | (g/kg) | (g/kg) | (%) | (%) | (%) |
| Rosmarinic | 23.51 | 51.00 | 73.58 | 98.2 | | |
| acid | | 25.50 | 49.38 | 101.5 | 98.7 | 2.6 |
| | | 12.75 | 35.81 | 96.5 | | |
| Oleanolic | 0.75 | 1.80 | 2.47 | 95.7 | | |
| acid | | 0.90 | 1.65 | 99.7 | 99.3 | 3.5 |
| | | 0.45 | 1.21 | 102.6 | | |
| Ursolic | 1.36 | 3.00 | 4.24 | 96.1 | | |
| acid | | 1.50 | 2.77 | 94.3 | 97.8 | 4.6 |
| | | 0.75 | 2.13 | 102.9 | | |

### Distribution of Rosmarinic Acid, Oleanolic Acid and Ursolic Acid in Different Mentha Species

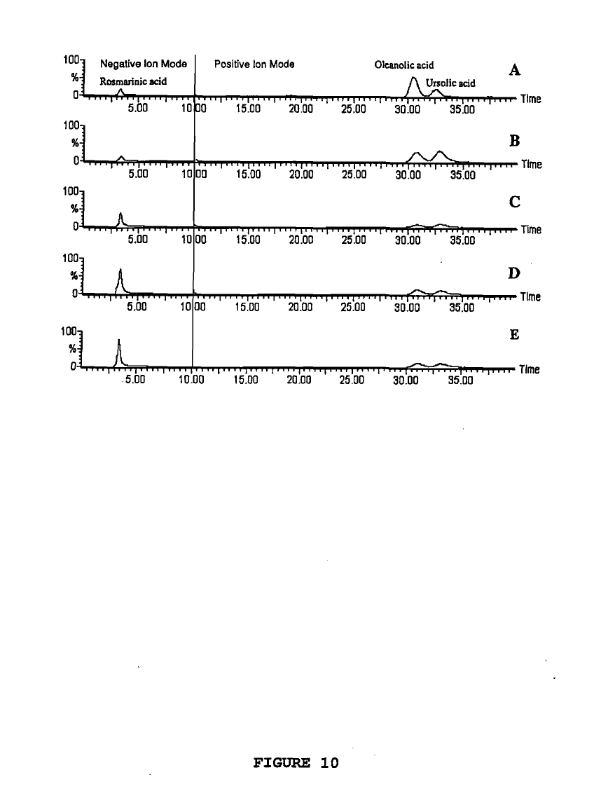

The use of species or varieties that display superior phytochemical content relative to the bioactivity can help to ensure the quality of botanicals or raw materials to the dietary supplement industry. In this invention, the inventors comparatively examined a wide range of mint species and sources of mints within many of the species in order to first ask whether there is genetic variation in the accumulation or expression of the polyphenols in mint; and if so, to then identify which sources may be the highest accumulators. Using this approach, the inventors hypothesized that several species would be significantly higher than others. Thus, such results could help to guide further collections made to develop improved sources of specific phenolic acids. The inventors also hypothesized that variation would be noted and that plant sources may vary in the specific targeted anti-inflammatory constituents while as an aggregate group or sum total the inventors may not observe such differences. By sampling the mint collection when all grown under the identical environmental conditions, the differential genetic expression (under the defined environmental condition) is easier to observe. As such, all plants were cultivated under identical field conditions. During the harvest season, 35 mints belonging to 9 different Mentha species were collected and dried under low temperature (32° C.), of which 25 mints were then quantitatively analyzed for rosmarinic acid, oleanolic acid and ursolic acid contents based on the LC/MS/MS method which the inventors developed (FIG. 10). The average rosmarinic acid content was 10.34 mg/g on dry weight basis across the 25 mints, with RSD=58.6%. The most rosmarinic acid-rich sample was Green Curly Mint (M. x piperita), followed by Spearmint (M. spicata). The contents of oleanolic acid and ursolic acid were much lower, both within the range of 0.12-5.11 mg/g in all samples (Table 5). The mean content levels of oleanolic acid and ursolic acid were calculated as 0.84 mg/g (RSD=79.6%) and 2.02 mg/g (RSD=78.6%), respectively. For most of the mints, the concentration of ursolic acid was 2 to 3 times higher than that of oleanolic acid, while this trend was inversed for Japanese Field Mint (M. Canadensis). In Japanese Field Mint, the oleanolic acid content (2.41 mg/g) was much higher than ursolic acid (0.54 mg/g). The Australian Mint (M. x gracilis) was characterized as that these two triterpenoid acids were almost at the same concentration level (0.43 mg/g versus 0.42 mg/g), whereas the concentration ratio of ursolic acid to oleanolic acid may reach to as high as 5.7 for Hajek Mint (M. longifolia). The high triterpenoid acid-rich samples include Grapefruit Mint (M. x piperita), Egyptian Mint (M. x villosa), Hillary's Sweet Mint (M. aquatica x M. suaveolens) and Apple Mint (M. x villosa). The inventors also noticed that the concentration variation of these three compounds was not limited among different species, but also observed striking differences, as expected within the species between the varieties and accessions. The genetic expression and influence in the different Mentha species as well as variety are critical components to consider in the standardization of anti-inflammatory agents from mint

TABLE 5
Anti-inflammatory Acid Contents in Different Sources of Mentha spp.
(Milligrams per Gram of Dry Matter)

| | | Rosmarinic | Oleanolic | Ursolic |
|---|---|---|---|---|
| Sample name | Latin name | acid | acid | acid |
| Peppermint | M. × piperita | 6.69 ± .38 | 0.79 ± .06 | 2.37 ± 0.09 |
| Spearmint | M. spicata | 23.51 ± 0.69 | 0.75 ± 0.05 | 1.36 ± 40.09 |
| Lavender | M. aquatica | 9.52 ± 0.64 | 0.12 ± 0.01 | 0.54 ± 0.03 |
| Mint | | | | |
| Persian Mint^{a} | M. × piperita | 14.56 ± 0.52 | 0.27 ± 0.01 | 0.67 ± 0.02 |
| Chewing | M. × piperita | 15.55 ± 0.21 | 0.60 ± 0.01 | 2.01 ± 0.08 |
| Gum Mint | | | | |
| Orange Mint | M. aquatica | 14.60 ± 0.40 | 0.30 ± 0.00 | 0.85 ± 0.01 |
| Apple Mint | M. × villosa | 12.21 ± 0.61 | 2.02 ± 0.03 | 4.78 ± 0.04 |
| Austrian | M. × gracilis | 11.37 ± 40.62 | 0.43 ± 0.03 | 0.42 ± 0.04 |
| Mint | | | | |
| Balsam Tea | M. × piperita | 3.73 ± 0.26 | 0.45 ± 0.02 | 1.39 ± 0.03 |
| Mint | | | | |
| Chocolate | M. × piperita | 6.44 ± 0.20 | 0.58 ± 0.02 | 1.54 ± 0.03 |
| Mint | | | | |
| Curly Mint | M. spicata | 9.33 ± 0.23 | 0.67 ± 0.01 | 1.74 ± 0.06 |
| Egyptian | M. × villosa | 7.66 ± 0.38 | 1.99 ± 0.03 | 5.11 ± 0.04 |
| Mint | | | | |
| Fuzzy | M. spicata | 19.76 ± 0.71 | 1.07 ± 0.04 | 2.93 ± 0.14 |
| Spearmint | | | | |
| Grapefruit | M. × piperita | 5.60 ± 0.26 | 2.00 ± 0.16 | 5.72 ± 0.36 |
| Mint | | | | |
| Green Curly | M. × piperita | 23.80 ± 0.42 | 0.72 ± 0.02 | 1.89 ± 0.06 |
| Mint | | | | |
| Hajek Mint^{b} | M. longifolia | 6.99 ± 0.42 | 0.45 ± 0.03 | 2.57 ± 0.18 |
| Hillary's | M. aquatica × M. | 5.79 ± 0.12 | 1.87 ± 0.03 | 5.04 ± 0.01 |
| Sweet Mint | suaveolens | | | |
| Hypocalyx | M. canadensis | 4.88 ± 0.35 | 1.25 ± 0.04 | 2.88 ± 0.08 |
| Mint | | | | |
| Japanese | M. canadensis | 1.75 ± 0.00 | 2.41 ± 0.03 | 0.54 ± 0.02 |
| Field Mint | | | | |
| Lime Mint | M. aquatica × M. | 2.99 ± 0.12 | 0.54 ± 0.02 | 1.78 ± 0.03 |
| | suaveolens | | | |
| Regular Mint | M. spicata | 10.14 ± 0.18 | 0.51 ± 0.01 | 1.41 ± 0.08 |
| Scotch Mint | M. × gracilis | 16.91 ± 0.30 | 0.21 ± 0.00 | 0.39 ± 0.04 |
| Todd | M. × piperita | 7.09 ± 0.26 | 0.45 ± 0.01 | 1.23 ± 0.08 |
| Mitcham | | | | |
| Mint | | | | |
| Variegated | M. × piperita | 9.94 ± 0.50 | 0.19 ± 0.00 | 0.50 ± 0.04 |
| Mint | | | | |
| Water Mint | M. × smithiana | 7.18 ± 0.40 | 0.53 ± 0.02 | 0.92 ± 0.02 |

^{a}Persian Mint was named by us at Rutgers to give respect to the purported origin of this unusual mint found in San Francisco by those of Persian background and used in cooking and salads. The mint was found and originally sent to Michigan State University where university researchers had evaluated it in the field and found the essential oils to be unacceptable relative to commercial traditional mint. They graciously gave this un-named mint to Prof. Simon and since then Rutgers has been working with it as a potential new culinary herb for those consumers seeking a more exotic less known mint with unusual aroma and flavor. This line has been named informally as Persian Mint and is also not commercially available.
^{b}Hajek mint refers to the commercial farm from which the materials was sourced from the commercial farm of Bob Hajek, Indiana. This material is also not commercially available.

### Characterization of the Flavonoids in Mints

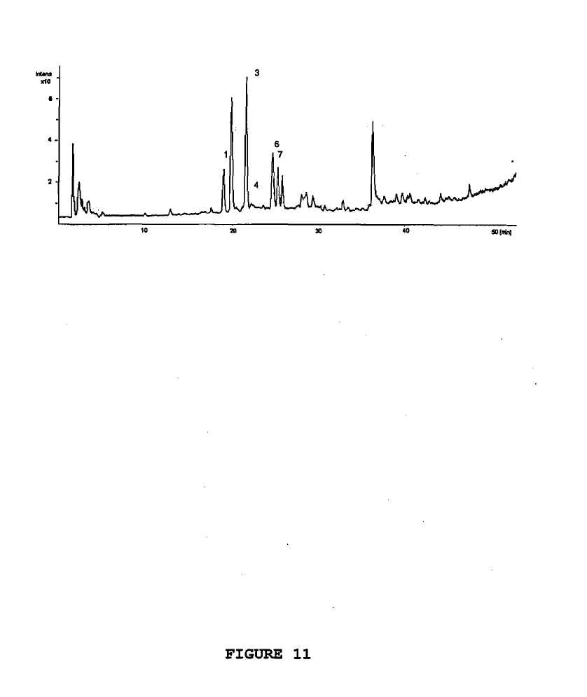

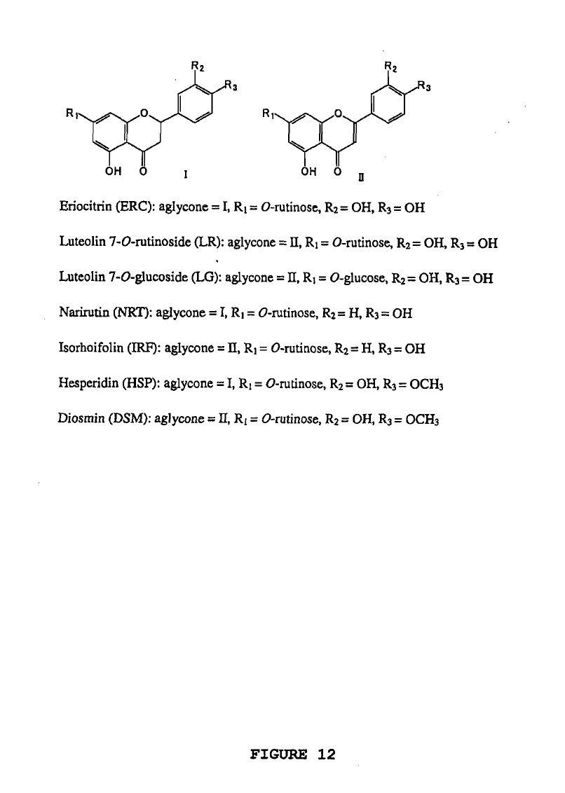

HPLC/MS coupled with a photodiode array detector (PAD) enabled us to obtain the UV and MS spectra for flavonoids on chromatograms. Wherever possible, the inventors compared the data with reported literature, and sometimes with the spectra of reference standards to check the identification and purity of each peak. Under positive mode, at the energy level of 60%, the flavonoids showed strong molecular ions [M+H]⁺ and/or [M+Na]⁺, and fragment peaks, including [M−glucose+H]⁺, [M−rhamnose+H]⁺ and [M−rhanmose−glucose+H]⁺ offering useful information to identify the sugar moieties. The neutral loss of m/z 146 (rhamnose) along with m/z 308 (rhamnose+glucose) is a typical MS fragmentation pattern for rutinosides. The inventors observed this fragmentation pattern for many peaks of chromatograms. According to the identification of rutinosides in Mentha species in literature, these compounds were tentatively determined as eriocitrin (peak 1), luteolin 7-O-rutinoside (peak 2), narirutin (peak 4), isorhoifolin (peak 5), hesperidin (peak 6), and diosmin (peak 7) (FIG. 11 and FIG. 12). The comparison with the UV spectra and retention times of the reference compounds on HPLC further confirmed our identification (Table 6). Peak 3 with molecular ion [M+H]⁺ at m/z 449 and fragmentation [M-glucose+H]⁺ at m/z 287 was identified as luteolin 7-O-glucoside, which was also reported by other researchers from mint (Guedon, D. J. and Pasquier, B. P., J. Agric. Food Chem. 1994, 42, 679-684; Kosar, M., et al., J. Agric. Food Chem. 2004, 52, 5004-5010).

TABLE 6
Peak Assignments of Flavonoids in Mint

| | | [M + H]⁺ | [M + Na]⁺ | fragmen- | | |
|---|---|---|---|---|---|---|
| Peak | tᵣ | MS | MS | tation | UV | compound |
| no. | (min) | (m/z) | (m/z) | ions (m/z) | λₘₐₓ | identification |
| 1 | 18.5 | 597 | | 289,328 | 284,328 | Eriocitrin |
| 2 | 20.3 | 595 | | 287,449 | 255,350 | Luteolin |
| 3 | 21.5 | 449 | | 287 | 254,350 | 7-O- |
| 4 | 22.2 | 581 | | 273,435 | 284,331 | rutinioside |
| 5 | 23.4 | 579 | | 271,433 | 267,338 | |
| 6 | 23.8 | 611 | 633 | 303,465 | 284,328 | |
| 7 | 24.4 | 609 | | 301,463 | 253,347 | |

By screening 29 Mentha spp. and sources within 8 species (some of mint samples were not screened due to the unavailability of harvesting from the research field), the inventors concluded that the flavonoids in mints were mostly glycolated with rutinoside. Some researchers reported the presence of free flavones such as eriodictyol and apigenin in Mentha species (Kosar, M., et al., J. Agric. Food Chem. 2004, 52, 5004-5010); however, they were hardly detected in our mint samples or detected at very low concentration levels. Acacetin 7-O-rutinoside was reported present in M. haplocalyx and M. arvensis (Lin, T., et al., Zhongguo Tianran Yaowu 2006, 4, 111-115; Oinone et al., 2006). The inventors detected this compound by HPLC/MS with fragmentation ion [M+H]⁺ at m/z 593 and fragmentations [M-rhamnose+H]⁺ at m/z 447 and [M-rhamnose-glucose+H]⁺ at m/z 285 in some M. piperita and M. aquatica varieties. Since our mint samples contain low concentration levels of acacetin 7-O-rutinoside, and no pure standard compound was commercially available, the quantification of this constituent was not performed and work with this compound was not continued.

### Quantitative Analysis of the Predominant Phenolic Constituents in Different Mentha Species

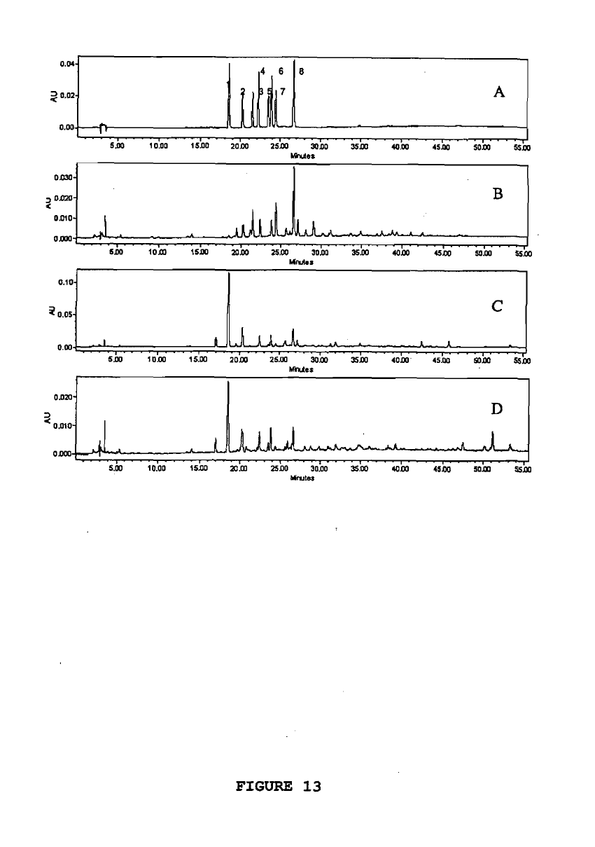

Some research groups have developed HPLC method to separate the phenolic compounds from several Mentha species. The purpose of this investigation was to further develop and validate an HPLC/UV quantitative method applicable for extensive quantitation of different mint varieties including some rare species. The inventors utilized a Luna C18 (2) column to achieve ideal resolution, particular with the separation of isorhoifolin, hesperidin and eriocitrin from other small peaks. A 2% isopropanol was added to the mobile phase to provide better peak shapes. This method was also successfully used for qualitative analysis in the research except 0.1% phosphoric acid was changed to 0.1% formic acid (volatile buffer) on LC/MS. Mints from eight Mentha species (M. x piperita, M. spicata, M. x gracilis, M. longifolia, M. Canadensis, M. x villosa, M. aquatica and M. aquatica x M. suaveolens) including the commercial varieties used in the production of mint oils, as well as many specialty mints were vegetatively propagated and field transplanted at the research farm, and further applied to quantitative analysis based on this HPLC method. Seven predominant flavonoids were efficiently separated and quantitated for all mint samples (FIG. 13). The analytes quantitated in the investigation cover the major phenolic constituents normally found in mints.

### Validation on the HPLC/UV Quantitative Method for Mint Samples

System precision was validated by conducting six replicated injections of a Chewing Gum Mint (M. x piperita) sample within the same working day. The RSDs (Relative Standard Deviations) of the peak areas for eriocitrin, luteolin-7-O-rutinoside, narirutin, isorhoifolin, hesperidin and diosmin were 0.50%, 0.44%, 1.00%, 2.38%, 0.57% and 1.70%, respectively; the RSDs of the retention times for these six compounds were calculated as 0.13%, 0.10%, 0.11%, 0.11%, 0.10% and 0.09%, respectively. The precision of the extraction procedure was also validated on the chewing gum mint. Four sample replicates, weighing about 100 mg, were extracted as described above in 25 mL of 70% methanol. An aliquot of each sample was then injected and quantitated. The average amounts of eriocitrin, luteolin-7-rutinoside, narirutin, isorhoifolin, hesperidin, diosmin and rosmarinic acid were 3.62% with a RSD of 0.50%, 1.51% with a RSD 0.50%, 0.13% with a RSD of 1.20%, 0.11% with a RSD of 2.50%, 0.51% with a RSD of 2.10% and 0.22% with a RSD of 1.56%, respectively. The recovery was evaluated by the spiking of five analytes, eriocitrin, luteolin-7-O-rutinoside, nadrutin, isorhoifolin and hesperidin approximately 100% of the expected values in a typical mint sample preparation. The recovered percentages were 98.9% for eriocitrin, 97.3% for luteolin-7-O-rutinoside, 100.4% for narirutin, 97.3% for isorhoifolin and 94.7% for hesperidin. In conclusion, the recommended method was reliable, consistent and precise for the quantitation of the predominant phenolic contents in a variety of mint samples.

### Distribution of the Phenolic Constituents in Various Mentha Species

Flavonoids, the major non-volatile phenolic compounds in mints, are distributed predominantly as glycolated forms, specifically flavonoid rutinosides. Rosmarinic acid, a typical phenolic acid present in many Lamiaceae family plants, is extensively present in all the tested mint samples, ranging from 0.22±0.16% to 1.41±0.36% through eight Mentha species on dry weight basis (Table 7 and Table 8). Peppermint and spearmint are the most cultivated crops in the United States for essential oil production. The other commercial mints may include but are not limited to Black Mitcham peppermint, Chinese mint, Japanese field mint, Todd Mitcham, and Scotch spearmint. Peppermint, Black Mitcham, black peppermint and Todd Mitcham are taxonomied as M. piperita. Along with other M. piperita varieties tested, namely variegated mint, chocolate mint, chewing gum mint, blue balsam mint, and green curly mint, the inventors found this widely grown species contained notably high concentrations of phenolics compared to other Mentha species, Eriocitrin was quantitated at 2.49±0.94% on dry weight basis in these six M. piperita varieties, followed by luteolin 7-O-rutinoside at 1.00±0.33%, and rosmarinic acid at 1.03±0.61%. The common commercial mint varieties were compared by the flavonoids (the sum of eriocitrin, luteolin 7-O-rutinoside, luteolin 7-O-glucoside narirutin, isorhoifolin, hesperidin, and diosmin) concentrations. Todd Mitcham mint showed the highest flavonoid content (5.84%), followed by black spearmint (3.62%) and spearmint (3.36%), all of which belong to M. piperita. The species M. spicata (spearmint, regular mint, Kentucky mint, fuzzy spearmint, curly mint and large-leaf spearmint) showed a different flavonoid profile, with significantly lower content of eriocitrin (0.06±0.09%) and a higher content of diosmin (0.49±0.20%) than that of M. piperita (0.15±0.07%). The other high phenolic content species noteworthy is M. aquatica (orange mint and lavender mint), with the eriocitrin content as 1.22±0.25%, luteolin 7-O-rutinoside as 0.71±0.15% and hesperidin as 0.66±0.21%. Based on this extensive survey of mint phenolic constituents, it can be concluded that many mint varieties particularly some commercial mints from M. piperita are rich source of bioactive phenolic compounds, and it would be worthwhile to consider the economical values of these non-volatile components from postdistillation materials.

TABLE 7
Flavonoids and Rosmarinic Acid Contents (g/100 g d.w.) in Different
Sources of Mint (Mentha spp.)^{a}

| Mint Varieties^{c} | ERC | LR | LG | NRT | IRF | HSP | DSM |
|---|---|---|---|---|---|---|---|
| Water Mint | 1.10 | 0.99 | —^{b} | — | 0.51 | 0.24 | 0.20 |
| Variegated Mint | 1.73 | 0.80 | 0 | 0.02 | 0.153 | 0.90 | 0.28 |
| Todd Mitcham | 3.73 | 1.40 | 0.0 | 0.13 | 0.10 | 0.36 | 0.12 |
| Mint | | | | | | | |
| Peppermint | 2.01 | 0.85 | — | — | 0.11 | 0.29 | 0.10 |
| Orange Mint | 1.39 | 0.81 | 0.04 | 0.15 | 0.46 | 0.80 | 0.35 |
| Lime Mint | 0.65 | 0.38 | — | 0.03 | 0.14 | 0.20 | 0.07 |
| Lavender Mint | 1.04 | 0.60 | — | — | 0.31 | 0.51 | 0.27 |
| Green Curly Mint | 1.20 | 0.57 | — | 0.03 | 0.08 | 0.55 | 0.16 |
| Egyptian Mint | 0.51 | 1.11 | — | — | 0.06 | 0.23 | 0.18 |
| Eau de Cologne | 1.16 | 0.78 | — | 0.03 | 0.30 | 0.50 | 0.37 |
| Mint | | | | | | | |
| Chocolate Mint | 3.28 | 1.17 | — | 0.03 | 0.13 | 0.31 | 0.12 |
| Chewing Gum mint | 3.62 | 1.51 | — | 0.13 | 0.11 | 0.51 | 0.22 |
| Blue Balsam Tea | 1.94 | 0.97 | — | — | 0.12 | 0.21 | 0.08 |
| Mint | | | | | | | |
| Black Peppermint | 2.40 | 0.74 | — | 0.02 | 0.09 | 0.28 | 0.09 |
| Apple Mint | 0.20 | 0.47 | 0.05 | — | 0.09 | 0.11 | 0.34 |
| Pineapple Mint | — | 0.12 | 0.05 | — | — | — | — |
| Spearmint | 0.03 | 0.23 | — | — | — | 0.19 | 0.69 |
| Regular Mint | 0.25 | 0.10 | — | — | 0.16 | 0.19 | 0.68 |
| Kentucky Mint | 0.03 | 0.48 | 0.06 | — | 0.17 | 0.29 | 0.47 |
| Hyplocalyx Mint | — | 0.25 | 0.05 | — | — | 0.22 | 0.23 |
| Horse Mint | 0.17 | 0.36 | 0.09 | 0.03 | 0.14 | 0.12 | 0.10 |
| Himalayan Silver | 0.06 | 0.24 | 0.05 | — | — | 0.13 | 0.08 |
| Mint | | | | | | | |
| Hillary's Sweet | 0.35 | 0.40 | — | — | 0.31 | 0.21 | 0.18 |
| Mint | | | | | | | |
| Fuzzy Spearmint | 0.03 | 0.20 | 0.30 | — | — | 0.16 | 0.56 |
| Curly Mint | 0.03 | 0.08 | — | — | — | 0.37 | 0.31 |
| Scotch Spearmint | — | — | — | — | — | 0.46 | 0.15 |
| Persian Mint | — | — | — | — | — | 1.67 | 0.49 |
| Large Leaf | — | 0.10 | — | — | — | 0.13 | 0.21 |
| Spearmint | | | | | | | |
| Japanese Field | — | — | — | — | — | 0.42 | 0.17 |
| Mint | | | | | | | |
| Grapefruit Mint | 0.05 | 0.10 | — | — | 0.04 | 0.62 | 0.41 |
| Chinese Mint | — | — | — | — | — | 0.18 | 0.61 |
| Austrian Mint | 0.02 | 0.09 | — | — | 0.08 | 0.86 | 0.73 |
| Ginger Mint | — | — | — | — | — | 0.08 | 0.93 |
| Bergamot Mint | — | — | — | — | — | | 0.27 |
| Hajek Mint | — | 0.14 | — | — | 0.12 | 0.07 | 0.23 |

^{a}Abbreviations: ERC, eriocitrin; LR, luteolin 7-O-rutinoside; LG, luteolin 7-O-glucoside; NRT, narirutin; IRF, isorhoifolin; HSP, hesperidin; DSM, diosmin.
^{b}Not detectable.
^{c}Mentha species listed in Table 5 and also in Table 8.

TABLE 8
Flavonoids and Rosmarinic Acid Contents (g/100 g d.w.) Summarized as
Different Mint (Mentha spp.) Species^{a}

| Species^{b} | ERC | LR | LG | NRT | IRF | HSP | DSM |
|---|---|---|---|---|---|---|---|
| Mentha x piperita | 2.49 ± 0.94 | 1.00 ± 0.33 | ND | 0.05 ± 0.05 | 0.11 ± 0.02 | 0.43 ± 0.22 | 0.15 ± 0.07 |
| Mentha spicata | 0.06 ± 0.09 | 0.20 ± 0.15 | 0.06 ± 0.12 | ND | 0.06 ± 0.09 | 0.22 ± 0.09 | 0.49 ± 0.20 |
| Mentha x gracilis | ND | 0.03 ± 0.05 | ND | ND | 0.03 ± 0.05 | 0.44 ± 0.43 | 0.38 ± 0.31 |
| Mentha longifolia | 0.08 ± 0.09 | 0.25 ± 0.11 | 0.05 ± 0.05 | 0.01 ± 0.02 | 0.09 ± 0.08 | 0.11 ± 0.03 | 0.14 ± 0.08 |
| Mentha | ND | 0.08 ± 0.14 | 0.02 ± 0.03 | ND | ND | 0.27 ± 0.13 | 0.34 ± 0.24 |
| canadensis | | | | | | | |
| Mentha x villosa | 0.36 ± 0.22 | 0.79 ± 0.45 | 0.03 ± 0.04 | ND | 0.08 ± 0.02 | 0.17 ± 0.08 | 0.26 ± 0.11 |
| Mentha aquatica | 1.22 ± 0.25 | 0.71 ± 0.15 | 0.02 ± 0.03 | 0.08 ± 0.11 | 0.39 ± 0.11 | 0.66 ± 0.21 | 0.31 ± 0.06 |
| Mentha aquatica x | 0.50 ± 0.21 | 0.39 ± 0.01 | ND | 0.02 ± 0.02 | 0.23 ± 0.12 | 0.21 ± 0.01 | 0.13 ± 0.08 |
| M. suaveolens | | | | | | | |

^{a}Abbreviations: ERC, eriocitrin; LR, luteolin 7-O-rutinoside; LG, luteolin 7-O-glucoside; NRT, narirutin; IRF, isorhoifolin; HSP, hesperidin; DSM, diosmin; RA, rosmarinic acid; ND, not detectable, b Values are expressed as mean ± SD generated from different varieties for the same species. M. x piperita was composed of peppermint, variegated mint, Todd Mitcham mint, chocolate mint, chewing gum mint, blue balsam mint, black peppermint and green curly mint, n = 8; M. spicata was composed of spearmint, regular mint, Kentucky mint, fuzzy spearmint, curly mint and large-leaf spearmint, n = 6; M. x gracilis was composed of Scotch mint, Austrian mint and bergamot mint, n = 3; M. longifolia was composed of horse mint, Himalayan silver mint and Hajek mint, n = 3; M. Canadensis was composed of Hypocalyx mint, Japanese field mint and Chinese mint, n = 3; M. x villosa was composed of Egyptian mint and apple mint, n = 2; M. aquatica was composed of orange mint and lavender mint, n = 2; M. aquatica x M. suaveolens was. composed of lime mint and sweet mint, n = 2. The other mints from Table 7 were not authenticated as to Mentha spp.

C. Recovery of Polyphenol and Triterpene Compounds from Oregano and Mint Plants

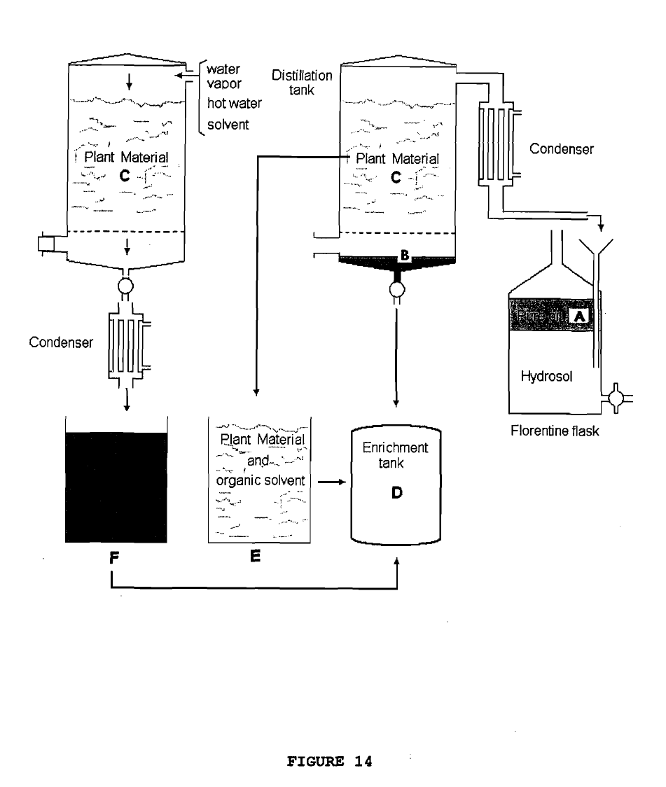

The present invention also provides a process that permits the procurement of water soluble polyphenols and triterpenes from aromatic plants both simultaneously and/or independent from the distillation of essential oils (or volatile aromatic oils from aromatic bearing plants such as peppermint, spearmint, lavender, basil, oregano, etc.). Using this model, with two representative plants, mint and oregano, the inventors both distilled the essential oil out of the plant using conventional distillation technologies (steam distillation, hydro-distillation, water and steam distillation) and found that following the distillation of each of the aromatic plants (mint and oregano) in which the essential oils were captured and would be processed as their own products of commerce as is done commercially; the leaves of each of these aromatic plants or essential oil crops retain approximately 60% of the phenols in the leaves after a normal distillation with the remainder or rest of the phenols which we found to be approximately 40% (but will range depending upon pressure and temperature of distillation, length of time of distillation, manner in which plant material is packed into the distillation retort or chamber, relative percent of moisture in the original essential oil bearing plants that are placed into the distillation chamber) is found in the water that collects and remains underneath the plant material at the bottom of the vessel. In distillation tanks there is usually a perforated plate of sorts that holds the plant material above where the entering steam and/or boiling water are found. The volatile aromatic oils are swept up out of the plant by the steam/water vapor and leave the distillation vessel and move into a condenser where the essential oils and water in vapor phase liquefy and then are separated. The water soluble polyphenols and triterpenes are not volatile and do not enter into that stream, but instead are found either to remain in the plant or accumulate at the bottom of the distillation tank (FIG. 14).

The present invention thus introduces a unique approach that is not done commercially but which will allow for both the capture and recovery of essential oil, as is done traditionally and commercially yet along with the capture and recovery of bio-active water soluble polyphenols and triterpenes that otherwise have traditionally entered the waste stream following the distillation of essential oil from aromatic or essential oil bearing plants.

Illustrative data for the recovery of the total phenols from mint and oregano are shown in Table 9, which, as a case study, indicates the presence and relative quantitation of the recovered water-soluble bioactive polyphenols in these plants. The method should be equally applicable to other aromatic plants.

TABLE 9
Quantitation of total phenols in aromatic plants extracted from the leaves and
flowers of mint and oregano^{a}

| | | | Total phenols in the | |
|---|---|---|---|---|
| | Total phenols in | | water that is | |
| | initial plant dry | Total phenols | remaining at the | Total amount |
| | material (before | in the wet leaves | bottom of the | after |
| | distillation)* | after distillation* | distillation vessel* | distillation* |
| Mint: Total amount | 3.9 g | 2.4 g | 1.6 g | 4 g |
| of polyphenols in | | | | |
| grams | | | | |
| Mint: Percentage | 100% | 61.5% | 41% | 102.6% |
| considering 100% | | | | |
| as the polyphenols | | | | |
| in the starting and | | | | |
| dry plant material | | | | |
| Oregano: Total | 5.5 g | 3.1 g | 2.3 g | 5.4 g |
| amount of | | | | |
| polyphenols in | | | | |
| grams | | | | |
| Oregano: | 100% | 56.7% | 42.0% | 98.7% |
| Percentage | | | | |
| considering 100% | | | | |
| as the polyphenols | | | | |
| in the starting and | | | | |
| dry plant material | | | | |

^{a}Total phenols measured both prior to distillation and from the wet leaves following distillation, and total phenols found in the distillation chamber underneath the plants following distillation.
*The technique used for quantifying the total phenols involves use of the Folin-Ciocalteu reagent (see Singleton V. L., et al., Methods in Enzimology 1999, 299, 152-178).

Although only the total phenols were measured as shown Table 9, it is clear that triterpenes as well as pigments and other bioactive compounds that are thermally stable elute into the water remaining on or below the distilled plant materials, and that it is the bioactive natural compounds found in this water stream that have been considered as a waste. In contrast, our system captures, enriches and recovers these biologically active compounds and converts such waste water into products that are of commercial value as dietary supplements, nutraceuticals, protodrugs, and functional food ingredients. In this waste water stream/pool we find bioactive natural products, such as rosmarinic acid, and other caffeic acid derivatives, and though not measured directly above (because the total phenol test does not quantify triterpenes), the water soluble triterpenes, including but not limited to oleanolic acid and ursolic acid, are also recovered in high percentages, as are the total polyphenols.

This patent process allows for the recovery of these bioactive water soluble polyphenols and triterpenes by converting what was waste (the spent plant materials following distillation or extraction of the essential oil from the plants (be it leaves, flowers, roots, bark, or any plant tissues singly or combined) which was and is traditionally then added back into the soil as compost, green manure, feed to animals, used in other manners except for the recovery of additional industrial chemicals. In addition, the recovery and enrichment of the water-remaining in association with the spent plant material as well as that at the bottom of the distillation chamber (also called retort, tank, tub, or container) was and is usually dumped/discarded and released as waste water. This water stream is distinctly different than the hydrosol or distillate water that separates from the essential oil after condensing but rather is water from the stream that never enters into the condenser but remains and is found with the spent or distillated plant material, under the plant material, and/or under a physical perforated removable floor or shelf upon which the plant material is placed. No claims are being made on the hydrosol or distillate water.

### EXAMPLES

### A. Analysis of Oregano Samples

The solvents methanol (HPLC-grade), hexane, ethyl acetate (EtOAc) and acetone (HPLC-grade) used for extraction and chromatography were purchased from Fisher Scientific Co. (Fair Lawn, N.J.). HPLC-grade water was prepared using a Millipore Milli-Q purification system (Millipore Corp., Bedford, Mass.) and used for preparing all solutions. The standard compound rosmarinic acid, HPLC buffers formic acid and ammonium hydroxide were procured from Sigma Chemical Co. (St. Louis, Mo.). The Syrian oregano (O. syriacum) varieties (SO1-SO5 and SO7) were purchased from Lebanon as dry aerial parts and directly used for HPLC analysis. GO2 (O. vulgare ssp. hirtum), SO6 (O. syriacum) and a Cuban oregano (Plectranthus amboinicus) variety were procured as seeds from Richters Herbs (Goodwood, Ontario, Canada) and propagated in the Department of Plant Biology and Pathology greenhouses, School of Environmental and Biological Sciences, Rutgers University. The rest of Greek oregano (O. vulgare ssp. hirtum) and European oregano (O. vulgare) varieties were collected as part of our ongoing plant breeding program in the New Use Agriculture and Natural Plant Products Program at Rutgers University and vegetatively transplanted into the Rutgers Snyder Research and Extension Farm in Pittstown, N.J., with parent plants being maintained and grown in the Rutgers greenhouse. This larger germplasm and breeding collection had been growing as a perennial crops with the plots being maintained using drip irrigation, and grown on raised beds, harvested once/year at full flowering. The plant aerial part was manually collected from all live plants at full flowering, and placed into a large-scale forced air drier at 40° C. for 2 weeks before any analytical study. All oregano varieties were microscopically authenticated by Dr. James Simon and deposited in Rutgers botanical products library for future reference.

### Example 1

### Isolation and Identification of Oleanolic Acid and Ursolic Acid from Oregano

The standard compounds oleanolic acid and ursolic acid were isolated from oregano samples (O. vulgare ssp. hirtum) and used for preparation of calibration standards. The dried oregano leaves (100 g) were extracted three times with ethanol and concentrated to dryness under reduced pressure. The residue was loaded to a silica gel (130-270 mesh) column and eluted by hexane-EtOAc (1:1), EtOAc, EtOAc-acetone (1:1) and acetone in sequence. A total of 16 fractions were collected, and the second fraction containing oleanolic acid and ursolic acid was further subjected to a preparative HPLC separation. The compounds oleanolic acid and ursolic acid were then purified using a Varian C 18 preparative column (250×41.4 mm, 8 μm) eluted with methanol-water (8:2). The structures of the two triterpenoid acids were elucidated by NMR and MS analysis.

### Example 2

### Cell Culture

RAW 264.7 cells, derived from murine macrophages, were procured from the American Type Culture Collection (Rockville, Md.). The cells were cultured in RPMI-1640 (without phenol red) supplemented with 10% endotoxin-free, heat-inactivated fetal calf serum (GIBCO, Grand Island, N.Y.), 100 units/mL penicillin, and 100 mg/mL streptomycin. When the RAW 264.7 cells reached a density of 2-3×10⁶ cells/mL, they were activated by incubation in the medium containing E. coli LPS (lipopolysaccharide, 100 ng/mL). Various concentrations of test compounds dissolved in DMSO (dimethylsulfoxide) were combined together with LPS. The cells were treated with 0.05% DMSO as vehicle control.

### Example 3

### Nitrite Assay

The RAW 264.7 cells were treated with various compounds and LPS or LPS only. The supernatants were harvested and the amount of nitrite, an indicator of NO synthesis, was measured using the Griess reaction. Briefly, supernatants (100 μL) were mixed with the same volume of Griess reagent (1% sulphanilamide in 5% phosphoric acid and 0.1% naphthylethylenediamine dihydrochloride in water) in duplicate on 96-well plates. After incubation at room temperature for 10 min, absorbance at 570 nm was measured with the ELISA reader (Thermo Labsystems, Multiskan Ascent, Finland).

### Example 4

### Western Blotting

The total proteins, isolated from cells after treatment with test compounds for 24 h, were extracted via addition of 200 μL of gold lysis buffer (50 mM Tris-HCl, pH 7.4; 1 mM NaF; 150 mM NaCl; 1 mM EGTA; 1 mM phenylmethanesulfonyl fluoride; 1% NP-40; and 10 μg/mL leupeptin) to the cell pellets on ice for 30 min, followed by centrifugation for 30 min at 4° C. The cytosolic fraction (supernatant) proteins were measured by Bio-Rad Protein Assay (Bio-Rad Laboratories, Munich, Germany). The samples (50 μg of proteins) were mixed with 5-fold sample buffer containing 0.3 M Tris-HCl (pH 6.8), 25% 2-mercaptoethanol, 12% sodium dodecyl sulfate (SDS), 25 mM EDTA, 20% glycerol, and 0.1% bromophenol blue. The mixtures were boiled at 100° C. for 5 min and then subjected to 12% SDS-polyacrylamide minigels for electrophoresis at a constant current of 20 mA. The proteins on the gel were electrotransferred onto an immobile membrane (PVDF; Millipore Corp., Bedford, Mass.) with transfer buffer composed of 25 mM Tris-HCl (pH 8.9), 192 mM glycine, and 20% methanol. The membranes were blocked with blocking solution containing 20 mM Tris-HCl, and then immunoblotted with primary antibodies including iNOS, β-actin, and COX-2 (Transduction Laboratories, Lexington, Ky.). The blots were rinsed three times with PBST buffer followed by incubating with 1:5000 dilution of the horseradish peroxide (HRP)-conjugated secondary antibody (Zymed Laboratories, San Francisco, Calif.). The transferred proteins were visualized with an enhanced chemiluminescence detection kit (ECL; Amersham Pharmacia Biotech, Buckinghamshire, UK).

### Example 5

### Preparation of Calibration Standards for HPLC Analysis

The stock solution was prepared by dissolving about 25 mg of each standard, rosmarinic acid, oleanolic acid and ursolic acid, in 45 mL methanol in a 50 mL volumetric flask. After sonication for 20 min, the flask was allowed to cool to room temperature and filled to volume with the diluent. Calibration curves were established on 15 data points by diluting the stock solution to cover the expected concentration range for rosmarinic acid, oleanolic acid and ursolic acid across all the oregano samples. The linearity range of the calibration curves was found to be 390 ng/mL-100 μg/mL for rosmarinic acid, 49 ng/mL— 25 μg/mL for oleanolic acid, and 24 ng/mL-25 μg/mL for ursolic acid.

### Example 6

### Analytical Instruments

Chromatographic analysis was performed on a Waters 2695 HPLC system (Waters Corp., Milford, Mass.) equipped with an auto-sampler, quaternary pump system, thermostatted column compartment, degasser and Millennium 3.2 software. Separation was achieved by using a tandem column system: a Synergi 50×4.6 mm, i.d. 4 μm, Polar-RP column (Phenomenex Inc., Torrace, Calif.) and downstream a Microsorb 100×4.6 mm, i.d. 3 μm, C18 column (Varian Inc., Palo Alto, Calif.). Mass spectrometer used in this research was a triple stage quadrupole Quattro II (Micromass Co., Altrincham, UK) equipped with the orthogonal Z-spray electrospray ionization (ESI) interface and the acquisition data processor Masslynx 3.4 software.

### Example 7

### Sample Preparation

All dried oregano samples were first finely ground with a coffee grinder. About 25 mg powder was accurately weighed from each sample and placed into a 50 mL volumetric flask, and about 45 mL of methanol was added. Each sample was sonicated for 20 min and allowed to cool to room temperature, and then filled to volume with the diluent. The extract was transferred to a centrifuge tube and centrifuged at 12,000 rpm for 2 min to obtain a clear solution and filtered through a 0.45 μm filter for HPLC analysis. The recoveries were validated by spiking each sample with known quantities of the standard compounds, rosmarinic acid, oleanolic acid and ursolic acid to approximately 100%, 75% and 50% of the expected values in the oregano samples and then extracting according the same extraction method described above.

### Example 8

### Mass Spectrometry Conditions

The ESI source of the Quattro II was operated with nitrogen serving as the nebulizing gas (10 L/h) and curtain gas (500 L/h). The source temperature was set at 120° C. and the desolvation temperature was held at 350° C. Full-scan mass spectra were obtained with a scan time of 2 sec and inter-scan delay of 0.1 sec, operating in negative ion mode for rosmarinic acid and positive ion mode for oleanolic acid and ursolic acid. For quantitation, a selection of m/z values corresponding to rosmarinic acid (m/z 359, [M−H]⁻), oleanolic acid (m/z 479, [M+Na]⁺) and ursolic acid (m/z 479, [M+N]⁺) were monitored by using the instrument in the SIM mode with a dwell time of 1 sec and inter-channel delay of 0.03 sec. The mass spectrometer was set for two time segments: 0-10 min for the detection of rosmarinic acid, where the SIM was carried out in the negative ion mode with the capillary voltage at 3.0 V, cone voltage at 40 V and extractor voltage at 5 V; and 10-40 min for the detection of oleanolic acid and ursolic acid, and the instrument was set in positive mode with the capillary voltage, cone voltage and extractor voltage at 3.2 V, 45 V and 8 V, respectively.

### B. Analysis of Mint Samples

### Example 9

### HPLC Analysis

The mobile phase for chromatographic separation consisted of solvent A (5 mM ammonium formate in water, pH 7.4, adjusted with ammonium hydroxide) and solvent B (5 mM ammonium formate in 90% methanol, pH 7.4) under an isocratic condition (13.5% solvent A and 86.5% solvent B) at a flow rate of 0.8 mL/min. One-fifth of the total effluent was split and injected into the electrospray LC/MS interface. The column compartment temperature was kept at 25° C. and the injection volume was 10 pt. Calibration curves were plotted using 1/x-weighted quadratic model for the regressing of peak area versus analyte concentration, resulting in equation of y=2.1655x+1157.9 (r²=0.999) for rosmarinic acid; y=183.14x−14038 (r²=1) for oleanolic acid; and y=270.22x+22627 (r²=0.999) for ursolic acid. All samples were run in duplicate.

### Example 10

### Plant Samples

A wide collection of live mint germplasm was procured from Purdue University and shipped over to Rutgers to be vegetatively propagated both in the greenhouse and later for field planting. This collection of 35 mints consisted of 9 species including the main species used in the commercial production of traditional peppermint, spearment and Japanese mint oils, plus a wide range of specialty mints grown as horticultural ornamentals and novelty varieties due to their unusual aromas and flavors. Several clonal materials of mints, not completely identified but found in commercial grower fields were also included for comparative purposes. All the mints once received at Rutgers (in 2001) were immediately rooted and grown in greenhouses under controlled conditions. Plant materials were then purposely increased by clonal or vegetative propagation until sufficient numbers of new plants were regenerated for field planting. In 2001, field plots were established for the entire mint collection and transplanted into the Rutgers Cooperative Research and Extension Center (Upper Deerfield, Cumberland, N.J.) in June, 2001, with parent plants being maintained and grown in the departmental greenhouse, School of Environmental and Biological Sciences, Rutgers University. The plants were cultivated under identical field conditions in single rows with 60 cm between plants and 2 m between beds. Field plots were maintained and harvested as needed during full flowering. Plots were hand weeded twice during the growing season and a Roterra cultivator maintained. No insecticides or fungicides were applied, and additional water was applied with a reel type irrigator. Harvesting was carried out by hand, and plant samples were dried at 40° C. for 2 weeks prior to chemical analysis. Given the wide array of mints and the difficulty in establishing and authenticating the varietal names and species, herbarium voucher specimens were collected for each mint in this invention. Herbarium voucher specimens of all 35 mints were then submitted for authentication to Dr. Arthur O. Tucker in Delaware State University, who microscopically identified and authenticated each accession in this invention. Voucher specimens of all entries were deposited into and are being stored at the Claude E Phillips Herbarium in Delaware State University, Dover, Del. standard compounds (rosmafinic acid, hesperidin and diosmin), and HPLC buffers (formic acid and ammonium hydroxide) were purchased from Sigma Chemical Co. (St. Louis, Mo.), luteolin 7-O-glucoside, luteolin 7-O-rutinoside, narirutin, isorhoifolin were purified from artichoke described before (53). Silica gel (130-270 mesh), RP-18 silica gel and Sephadex LH-20 (Sigma Chemical Co., St. Louis, Mo.) were used for column chromatography. Oleanolic acid and ursolic acid used as standards were isolated from oregano (Origanum vulgare spp. hirtum), another member of the Lamiaceae family plant, and structurally elucidated by NMR and MS analysis. The liquid nitrogen used for LC/MS analysis was high-purity nitrogen (99.999%) and from Airgas Co. (Salem, N.H.). ¹H-NMR and ¹³C-NMR spectra were obtained on the 200 MHz NMR spectroscopy (Varian Inc., Palo Alto, Calif.).

### Example 11

### Analytical Instruments

HPLC analysis was applied on a Waters 2695 system (Waters Corp., Milford, Mass.) equipped with an auto-sampler, quaternary pump system, thermostatted column compartment, degas ser and Millennium 3.2 software. Chromatographic separation was achieved by using a tandem column system: a Synergi 50×4.6 mm, i.d. 4 μm, Polar-RP column (Phenomenex Inc., Torrace, Calif.) and downstream a Microsorb 100×4.6 ram, i.d. 3 μm, C18 column (Varian Inc., Palo Alto, Calif.). Mass spectrometer used in this research was a triple stage quadrupole Quattro//instrument (Micromass Co., Altrincham, UK) equipped with the orthogonal Z-spray electrospray ionization (ESI) interface and the acquisition data processor Masslynx 3.4 software.

### Example 12

### Qualitative Analysis of Flavonoids by HPLC/UV/MS

The quantitative analysis utilized an Agilent 1100 Series LC/UV/MSD system (Agilent Technologies, Waldbronn, Germany) equipped with a quaternary pump, diode array and multiple wavelength detector, thermostatted column compartment, degasser, and electrospray source. The software was HP ChemStation, Bruker Daltonics 4.0 and Data analysis 4.0. The mobile phase used the same gradient procedure as the HPLC method described above except mobile phase A was 0.1% formic acid solution instead of 0.1% phosphoric acid. The eluent flow was a 2 to 1 stream splitting for the MSD detector. The electrospray-ionization mass spectrometer (ESI-MS) was operated under positive ion mode and optimized collision energy level of 60%, scanned from m/z 100 to 1000. ESI was conducted using a needle voltage of 3.5 kV. High-purity nitrogen (99.999%) was applied as drying gas with flow rate at 9 L/min Capillary temperature was 325° C., and nebulizer was set at 45 psi. The ESI interface and mass spectrometer parameters were optimized to obtain maximum sensitivity. The UV absorption spectra were recorded from 200 to 400 nm for all peaks.

### Example 13

### Calibration Standards for Anti-Inflammatory Content Analysis

The stock solution was prepared by dissolving approximate amount of 25 mg each standard, rosmarinic acid, oleanolic acid and ursolic acid, with 45 mL methanol in a 50 mL volumetric flask. The solution was sonicated for 20 min and allowed to cool to room temperature before filling to volume with the diluent. Calibration curves were established on 15 data points by diluting the stock solution to cover the expected concentration range for rosmarinic acid, oleanolic acid and ursolic acid in all mint samples. The linearity of the calibration curves was determined as 3.13 μg/mL-400 μg/mL for rosmarinic acid, 98 ng/mL-25 μg/mL for oleanolic acid, and 391 ng/mL-25 μg/mL for ursolic acid. 10 μL aliquot was used for HPLC analysis.

### Example 14

### Calibration Standards for Flavonoid Analysis

About 5 mg of each compound was accurately weighed into a 25 mL volumetric flask. 5 mL DMSO and 15 mL of 70% methanol was added and the flask was sonicated for 15 min. The flask was allowed to cool to room temperature and filled to full volume with 70% methanol solution. 5 mL of the above solution was transferred to a new 50 mL volumetric flask and diluted to the full Volume with 70% methanol. Calibration curves were established on six data points covering a concentration range of 0.25-250 μg/mL for eriocitrin, 0.224-224 μg/mL for luteolin-7-O-rutinoside, 0.172-172 μg/mL for luteolin-7-O-glucoside, 0.184-184 μg/mL for narirutin, 0.212-212 μg/mL for isorhoifolin, and 0.21-210 μg/mL for hesperidin, and 0.315-315 μg/mL for diosmin, with correlation coefficients of 1 for all seven compounds. 10 μL aliquot was used for HPLC analysis.

### Example 15

### Plant Sample Preparation for Anti-inflammatory Content Analysis

Air-dried mint samples (aerial part without flowers) were finely ground with a coffee grinder. About 25 mg plant powder was accurately weighed from each sample and placed into a 50 mL volumetric flask with about 45 mL of methanol added. The sample was sonicated for 20 min and allowed to cool to room temperature before filling to volume with the diluent. The extract was transferred to a centrifuge tube and centrifuged at 12,000 rpm for 2 min to obtain a clear solution, which was then filtered through a 0.45 μm filter and placed into sample vials for HPLC analysis. The validation on recoveries were carried out by spiking each sample with known quantities of standard compounds, rosmarinic acid, oleanolic acid and ursolic acid to approximately 200%, 100% and 50% of the expected values in the mint samples and then extracting together according to the same extraction method.

### Example 16

### Plant Sample Preparation for Flavonoid Analysis

The dried mint aerial part was finely ground with a coffee grinder. About 100 mg of powder was accurately weighed into a 25 mL volumetric flask. 20 mL of 70% methanol solution was added and the samples were sonicated for 30 min. The flasks were allowed to cool to room temperature and filled to the final volume with 70% methanol. Using a disposable syringe and 0.45 μm filter, the samples were filtered into HPLC vials for HPLC analysis.

### Example 17

### Quantitative Determination of Anti-inflammatory Constituents by LC/MS/MS Method

The mobile phase for chromatographic separation was composed of solvent A (5 mM ammonium formate in water, pH 7.4, adjusted with ammonium hydroxide) and solvent B (5 mM ammonium formate in 90% methanol, pH 7.4) under an isocratic condition (13.5% solvent A and 86.5% solvent B). The flow rate was set at 0.8 mL/min, and four-fifths of the total effluent was split before injecting into the electrospray LC/MS interface. The injection volume was 10 μL, and the column compartment temperature was maintained at 25° C. A plot of the area response ratio versus concentration resulted in calibration equation of y=0.196x−481.9 (r²=0.999) for rosmarinic acid; y=0.9444x−46.776 (r²=0.998) for oleanolic acid; and y=0.3776x−118.05 (r²=0.999) for ursolic acid. All samples were run in duplicate. The ESI source of the Quattro II uses nitrogen as the nebulizing gas (10 L/h) and curtain gas (500 L/h). The ion source temperature was kept at 120° C. and the desolvation temperature was set at 350° C. Full-scan mass spectra were operated with a scan time of 2 sec and inter-scan delay of 0.1 sec, under negative ion mode for rosmarinic acid and positive ion mode for oleanolic acid and ursolic acid. Quantitation was performed by LC/MS/MS in the multiple reaction monitoring (MRM) mode. Rosmarinic acid was monitored with a selection of m/z 359 as the parent ion and m/z 161 as the daughter ion; oleanolic acid and ursolic acid were detected by choosing m/z 439 as the parent ion and m/z 203 as the daughter ion. The instrument was set with a dwell time of 1 sec and inter-channel delay of 0.03 sec. The mass chromatographic monitoring was divided into two time segments: 0-10 min for the detection of rosmarinic acid, where the MRM was carried out in the negative ion mode with the capillary voltage at 3.0 V, cone voltage at 40 V and extractor voltage at 5 V; and 10-40 min for the detection of oleanolic acid and ursolic acid, where the instrument was set in positive mode with the capillary voltage, cone voltage and extractor voltage at 3.2 V, 45 V and 8 V, respectively.

### Example 18

### Quantitative Determination of Flavonoids and Rosmarinic Acid by HPLC/UV Method

Quantitative HPLC analysis was performed on a Waters HPLC system 2695 equipped with an auto-sampler, a quaternary pump system, a photodiode array detector 2996 and Millennium 3.2 software. Separation used a Luna C18 (2) column, 5 μm, 250×4.6 mm i.d. (Phenomenex Inc., Torrace, Calif.). The column temperature was set to 30° C. and the mobile phase included water (containing 0.1% phosphoric acid, solvent A), acetonitrile (solvent B), and isopropanol (solvent C) in a gradient system. The gradient ran as follows: 0 min, 88% solvent A, 10% B and 2% C; 30 minutes, 68% A, 30% B and 2% C; 50 minutes, 38% A, 60% B and 2% C; 60 minutes, 38% A, 60% B and 2% C; the post running time is 10 min. The flow rate was 1.0 mL/min, injection volume was 10 μL and detection wavelength was set at 280 nm

### Example 19

### Qualitative Analysis by HPLC/UV/MS

Qualitative analysis utilized an Agilent 1100 Series LC/MSD system (Agilent Technologies, Waldbronn, Germany) equipped with a quaternary pump, diode array and multiple wavelength detector, thermostatted column compartment, degasser, and electrospray source. The software was HP ChemStation, Bruker Daltonics 4.0 and Data analysis 4.0. The mobile phase was the same as the HPLC method above except mobile phase A was 0.1% formic acid solution instead of 0.1% phosphoric acid. The flow was a 2 to 1 stream splitting for the MSD detector. The electrospray-ionization mass spectrometer (ESI-MS) was operated under positive ion mode and optimized collision energy level of 60%, scanned from m/z 100 to 1000. ESI was conducted using a needle voltage of 3.5 kV. High-purity nitrogen (99.999%) was applied as drying gas with flow rate at 9 L/min Capillary temperature was 325° C., and nebulizer was set at 45 psi. The ESI interface and mass spectrometer parameters were optimized to obtain maximum sensitivity. The UV absorption spectra were recorded from 200 to 400 nm for all peaks.

### C. Recovery of Polyphenol and Triterpene Compounds

### Example 20

### Recovery of Polyphenol and Triterpene Compounds from Spent Plant Material

After the distillation, the essential oil is recovered in one path, and then most of the water soluble polyphenols and triterpenes that are found in the leaves (60% of the original polyphenols in the plant materials before distillation), and/or flowers and/or flowering tops which include leaves, stems and flowers, and in the dark green water extract at the bottom of the vessel (40%) can be captured and enriched and then prepared into and as a separate product of commerce for which this technology of recovering the water soluble polyphenols and triterpenes is described. See areas C and B in FIG. 14 above. After the distillation of aromatic plants, the product of commerce (A), the essential oil is collected from a Florentine flask or similar separatory vessel that facilitates the separation of essential oil from the re-condensed water often by specific gravity. The distillate water is that below the oil layer and can be described as hydrosol or distillate water. This invention does not make any claim as to the oil and/or the distillate water as shown in the diagram FIG. 14 (the oil layer marked as ‘A’ or the aromatic water which is often is discarded. In this invention the inventors find and report that both industrially valuable polyphenols and also triterpenes are stable and can be recovered from the plant materials and/or associated water with the plant materials and in the water underneath the plant material in the retort or distillation chamber after the distillation process. These polyphenols and triterpenes with valuable anti-inflammatory activities are found in the dark green water extract found at the bottom of the distillation vessel (B) and in the spent plant material (C). We observed that the plant material (after distillation) contained 60% of the polyphenols of initial total amount of polyphenol found in the plant material before the distillation. The rest (40%) is found in the dark green water extract found in the bottom of the distillation vessel.

At D, a retort or chamber is used to collect the water which includes the water soluble polyphenols and triterpenes, then the water removed by standard methods (including but not limited to freeze drying, rotary evaporation, spin chromatography, differential boiling, spray drying, etc). The spent plant material (C) can then be further extracted with solvents (E) (including ethanol, butanol, and ethyl acetate) and then the polyphenols and triterpenes concentrated by the enrichment retort (D) and also later spray dried to make available in dry form. After the distillation process, another option is to supply a revert current of water vapor, hot water or solvent so as to wash and further extract the soluble polyphenols which are then condensed to obtain more amount of water extract B, that can then be processed to concentrate the polyphenols (D).

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. An anti-inflammatory composition derived from an oregano or mint plant or a species of the Lamiaceae family, the composition comprising at least one organic acid selected from the group consisting of rosmarinic acid, oleanolic acid and ursolic acid.

2. The anti-inflammatory composition of claim 1, wherein the oregano plant is a species selected from Origanum vulgare ssp. hirtum, Origanum vulgare, and Origanum syriacum.

3. (canceled)

4. The anti-inflammatory composition of claim 1, wherein the mint plant is a Mentha spp. selected from the group consisting of peppermint (M. x piperita), spearmint (M. spicata), lavender mint (M. aquatica), Persian mint (M. x piperita), chewing gum mint (M. x piperita), orange mint (M. aquatica), apple mint (M. x villosa), Austrian mint (M. x gracilis), balsam tea mint (M. x piperita), chocolate mint (M. x piperita), curly mint (M. spicata), Egyptian mint (M. x villosa), fuzzy spearmint (M. spicata), grapefruit mint (M. x piperita), green curly mint (M. x piperita), Hajek mint (M. longifolia), Hillary's sweet mint (M. aquatica x M. suaveolens), Hypocalyx mint (M. canadensis), Japanese field mint (M. canadensis), lime mint (M. aquatica x M. suaveolens), regular mint (M. spicata), Scotch mint (M. x gracilis), Todd Mitcham mint (M. x piperita), variegated mint (M. x piperita), and water mint (M. x smithiana).

5. A dietary supplement comprising the anti-inflammatory composition of claim 1.

6. The anti-inflammatory composition of claim 1, wherein rosmarinic acid, oleanolic acid and ursolic acid are present in a 2:1:2 ratio by weight.

7. A dietary supplement comprising the anti-inflammatory composition of claim 6.

8. A method for treating an inflammatory condition in a human or animal subject, the method comprising administration of a therapeutically effective amount of the anti-inflammatory composition of claim 1 to the subject.

9. The method of claim 8, wherein the subject is a horse and the inflammatory condition is equine rheumatic arthritis.

10. A method for isolation of an organic acid from an oregano or mint sample, the method comprising the steps of:
(a) extracting the oregano or mint sample with an organic solvent to obtain an organic extract and a solid residue;
(b) optionally separating the solid residue from the organic extract;
(c) evaporating the organic solvent from the organic extract to obtain a liquid residue enriched with the organic acid;
(d) isolating the organic acid from the liquid residue by silica gel or ion-exchange chromatography eluting with a gradient of organic solvents until the subject organic acid is eluted out in eluent fraction(s);
(e) evaporating the eluting solvents from the fraction(s) containing the subject organic acid obtained in step (d); and
(f) optionally repeating steps (d) and/or (e) until the subject organic acid is completely separated from other components of the liquid residue.

11. The method of claim 10, wherein the oregano or mint sample is in the form of a dry powder.

12. The method of claim 10, wherein the organic solvent(s) in the extracting step comprises a C₁-C₆ alcohol.

13. The method of claim 12, wherein the alcohol is methanol or ethanol.

14. The method of claim 10, wherein the method further comprises validation steps of (g) spiking the oregano or mint sample with a known quantity of the subject organic acid standard, and (h) measuring the total amount of the subject organic acid in the mixture.

15. The method of claim 10, wherein the organic acid is selected from the group consisting of rosmarinic acid, oleanolic acid and ursolic acid.

16. (canceled)

17. (canceled)

18. (canceled)

19. (canceled)

20. A method for recovering water-soluble polyphenol and/or triterpene compounds from an oregano or mint plant, comprising extracting the plant material with a hot water or water vapor to obtain an aqueous phase enriched by polyphenol and/or triterpene compounds and isolating the polyphenol and/or triterpene compounds from the enriched aqueous phase, wherein the extracting step optionally occurs in tandem with a distillation step.

21. The method of claim 20, further comprising a step of separating the enriched aqueous phase from an oil phase that may be formed.

22. The method of claim 20, wherein the extracting step further comprises extracting the plant material with a water-immiscible organic solvent to obtain an organic phase enriched with oil compounds and separating the polyphenol and/or triterpene enriched aqueous phase from the organic phase.

23. The method of claim 20, wherein the extracting step occurs in tandem with a distillation step, and wherein the distillation step removes volatile essential oils from the plant material.

24. The method of claim 23, wherein after distillation the spent plant material is further extracted with a revert current water vapor, hot water or solvent to obtain an additional amount of extract enriched with the polyphenol and/or triterpene compounds.

25. The method of claim 20, further comprising a step of quantifying the remaining content of the polyphenol and/or triterpene compounds in the plant material.

26. The method of claim 20, further comprising a step of quantifying the total amount of phenols remaining in the plant material by using the Folin-Ciocalteu method.

27. The method of claim 20, wherein the enriched aqueous solution is an aqueous waste from distillation.

28. The method of claim 20, wherein the isolating step comprises removal of water by evaporation, freeze drying, spin chromatography, differential boiling, or spray drying.

29. The method of claim 20, further comprising separation of the polyphenol and/or triterpene compounds by chromatography.

30. An anti-inflammatory composition comprising at least one of the polyphenol and triterpene compounds recovered according to the method of claim 20.
